(19) United States
(12) Patent Application Publication
(10) Pub. No.: US 2007/0053987 A1
(43) Pub. Date: Mar. 8, 2007

(54) Cross-linked polysacharide and protein matrices and methods for their preparation

(76) Inventors: Thomas Bayer, Tel-Aviv (IL); Arie Goldlust, Ness-Ziona (IL)

Correspondence Address: DANIEL J SWIRSKY, 55 REUVEN ST., BEIT SHEMESH, 99544, IL

(21) Appl. No.: 11/513,345

(22) Filed: Aug. 31, 2006

Related U.S. Application Data
(60) Provisional application No. 60/713,390, filed on Sep. 2, 2005.

(51) Int. Cl. A61K 48/00; A61K 38/22; A61K 38/18; A61K 38/14; C07H 5/06; C08B 37/00; A61K 9/14

(52) U.S. Cl. 424/488; 514/12; 514/44; 514/8; 536/53; 536/55.3

(57) ABSTRACT

Methods for preparing cross-linked polysaccharide matrices by cross-linking one or more amino group containing polysaccharides or amino-functionalized polysaccharides with reducing sugars and/or reducing sugar derivatives. The resulting matrices may include polysaccharide matrices and composite cross-linked matrices including polysaccharides cross-linked with proteins and/or polypeptides. Additives and/or cells may also be included in or embedded within the matrices. Various different solvent systems and reducing sugar cross-linkers for performing the cross-linking are described. The resulting matrices exhibit various different physical, chemical and biological properties.

### CROSS-REFERENCE TO RELATED APPLICATIONS

This application claims priority from and the benefit of U.S. Provisional Patent Application Ser. No. 60/713,390, filed on Sep. 2, 2005, entitled “CROSS-LINKED POLYSACCHARIDE MATRICES AND METHODS FOR THEIR PREPARATION” incorporated herein by reference in its entirety.

### FIELD OF THE INVENTION

The present invention relates generally to cross-linked polysaccharide based matrices and preparations and more particularly to a novel method for cross linking amino-polysaccharides and amino-functionalized polysaccharides using reducing sugars and their derivatives as cross-linking agents and to cross linked polysaccharide matrices and preparations formed by using this method.

### BACKGROUND OF THE INVENTION

The performance of hyaluronic acid-based or other amino-saccharide based products depends on the one hand on controlling their functional longevity within the host and on the other hand on the preservation of the biological properties of the native hyaluronic acid (HA) or other polysaccharide component. The functional longevity of the HA or other polysaccharide component depends on its capacity to resist specific enzymatic degradation by hyaluronidase or by any other polysaccharide degrading enzymes present in the host. This capacity is directly related to the number of intra-molecular and intermolecular cross-links within the HA or other polysaccharide based polymer. Typically, a higher number of cross-links results in a higher resistance to such enzymatic degradation.

Exemplary cross-linking agents of choice known in the art for cross-linking polysaccharides and/or derivatives of polysaccharides and/or artificially functionalized forms of polysaccharides have been bifunctional (or poly functional) linkers such as, for example 1,4 bunandioldiglycidyl ether, a variety of other synthetic bifunctional cross-linkers and other related non-physiological agents. These cross-linking agents react with amino groups or other functional groups of the polysaccharide molecule to form intermolecular cross-links. However, these harsh agents may have negative effects on the biocompatibility and biological activity of cross-linked polysaccharide-based bioproducts that may be caused by alterations in the conformation of the polysaccharide molecule and leaching out of the cross-linking agents. Thus, polysaccharide products cross-linked by non-physiological agents may exhibit some degree of antigenicity. Furthermore, localized inflammation and more complex systemic reactions including local swelling, pruritus, transient or long term erythema, oedema, granuloma formation, superficial necrosis urticaria and acneform lesions may be disadvantageous side effects in a small percentage of patients esthetically treated with of commercially existing cross-linked polysaccharide products.

Additionally, in the case when the products are formulated for injection in the form of suspensions, gels or emulsions, the use of artificial cross-linkers known in the art may not always allow the obtaining of cross-linked products having satisfactory resistance to enzymatic degradation in combination with desired rheological properties of the injectable preparation.

### SUMMARY OF THE INVENTION

There is therefore provided, in accordance with an embodiment of the present invention, a method for preparing cross-linked polysaccharides. The method includes reacting at least one polysaccharide selected from an amino-polysaccharide and/or an amino-functionalized polysaccharide and/or combinations thereof with at least one reducing sugar, to form a cross-linked polysaccharide.

Furthermore, in accordance with an embodiment of the invention, the at least one polysaccharide is selected from a naturally occurring amino-polysaccharide, a synthetic amino-polysaccharide, an amino heteropolysaccharide, an amino homopolysaccharide, amino-functionalized polysaccharides and derivatized forms and esters and salts thereof, amino-functionalized hyaluronic acid and derivatized forms and esters and salts thereof, an amino-functionalized hyaluronan and derivatized forms and esters and salts thereof, chitosan and derivatized forms thereof and esters and salts thereof, heparin and derivatized forms and esters and salts thereof, amino functionalized glycosaminoglycans and derivatized forms and esters and salts thereof, and any combinations thereof.

Furthermore, in accordance with an embodiment of the invention, the at least one reducing sugar is selected from an aldose, a ketose, a derivative of an aldose, a derivative of a ketose, a diose, a triose, a tetrose, a pentose, a hexose, a septose, an octose, a nanose, a decose, glycerose, threose, erythrose, lyxose, xylose, arabinose, ribose, allose, altrose, glucose, fructose, mannose, gulose, idose, galactose and talose, a reducing monosaccharide, a reducing disaccharide, a reducing trisaccharide, a reducing oligosaccharide, derivatized forms of oligosaccharides, derivatized forms of monosaccharides, esters of monosaccharides, esters of oligosaccharides, salts of monosaccharides, salts of oligosaccharides, maltose, lactose, cellobiose, gentiobiose, melibiose, turanose, trehalose, isomaltose, laminaribiose, mannobiose and xylobiose, glyceraldehyde, ribose, erythrose, arabinose, sorbose, fructose, glucose, D-ribose-5-phosphate, glucosamine, and combinations thereof.

Furthermore, in accordance with an embodiment of the invention, the at least one reducing sugar may be selected from a Dextrorotatory form of the at least one reducing sugar, a Laevorotatory form of the at least one reducing sugar and a mixture of Dextrorotatory and Laevorotatory forms of the at least one reducing sugar.

Furthermore, in accordance with an embodiment of the invention, the reacting comprises incubating the at least one polysaccharide in a solution including at least one solvent and at least one reducing sugar, to form the cross-linked polysaccharide.

Furthermore, in accordance with an embodiment of the invention, the solution is a buffered solution including at least one buffer.

Furthermore, in accordance with an embodiment of the invention, the solvent is an aqueous buffered solvent including at least one buffer for controlling the pH of the solution.

Furthermore, in accordance with an embodiment of the invention, the solvent is an aqueous solvent including at least one ionizable salt for controlling the ionic strength of the solution.

Furthermore, in accordance with an embodiment of the invention, the solvent(s) includes at least one solvent selected from the group consisting of an organic solvent, an inorganic solvent, a polar solvent, a non-polar solvent, a hydrophilic solvent, a hydrophobic solvent, a solvent miscible in water, a non-water miscible solvent and combinations thereof.

Furthermore, in accordance with an embodiment of the invention, the solvent includes water and at least one additional solvent selected from a hydrophilic solvent, a polar solvent, a solvent miscible in water and combinations thereof.

Furthermore, in accordance with an embodiment of the invention, the solvent is selected from the group consisting of water, phosphate-buffered saline, ethanol, 2-propanol, 1-butanol, 1-hexanol, acetone, ethyl acetate, dichloromethane, diethyl ether, hexane, toluene, and combinations thereof.

Furthermore, in accordance with an embodiment of the invention, the reacting includes adding at least one protein and/or polypeptide having cross-linkable amino groups to the at least one polysaccharide and the at least one reducing sugar to form a composite cross-linked matrix.

Furthermore, in accordance with an embodiment of the invention, the at least one protein and/or polypeptide having cross-linkable amino groups is selected from collagen, a protein selected from the collagen superfamily, extra-cellular matrix proteins, enzymes, structural proteins, blood derived proteins glycoproteins, lipoproteins, natural proteins, synthetic proteins, hormones, growth factors, cartilage growth promoting proteins, bone growth promoting proteins, intracellular proteins, extracellular proteins, membrane proteins, elastin, fibrin, fibrinogen and any combinations thereof.

Furthermore, in accordance with an embodiment of the invention, the collagen is selected from, native collagen, fibrillar collagen, fibrillar atelopeptide collagen, telopeptide containing collagen, lyophylized collagen, collagen obtained from animal sources, human collagen, mammalian collagen, recombinant collagen, pepsinized collagen, reconstituted collagen, bovine atelopeptide collagen, porcine atelopeptide collagen, collagen obtained from a vertebrate species, recombinant collagen, genetically engineered or modified collagen, collagen types I, II III, V, XI, XXIV, fibril-associated collagens types IX, XII, XIV, XVI, XIX, XX, XXI, XXII and XXVI, collagens types VIII and X, type IV collagens, type VI collagen, type VII collagen, type XIII, XVII, XXIII and XXV collagens, type XV and XVIII collagens, artificially produced collagen manufactured by genetically modified eukaryotic or prokaryotic cells or by genetically modified organisms, purified collagen and reconstituted purified collagen, particles of fibrillar collagen, fibrillar reconstituted atelopeptide collagen, collagen purified from cell culture medium, collagen derived from genetically engineered plants, fragments of collagen, proto-collagen and any combinations thereof.

Furthermore, in accordance with an embodiment of the invention, the reacting includes adding at least one additive to the at least one polysaccharide and theat least one reducing sugar to form a cross-linked matrix containing at least one additive.

Furthermore, in accordance with an embodiment of the invention, the at least one additive is selected from pharmaceuticals, drugs, proteins, polypeptides, anesthetic agents, anti-bacterial agents, anti-microbial agents, anti-viral agents, anti-fungal agents, anti-mycotic agents, anti-inflamatory agents, glycoproteins, proteoglycans, glycosaminoglicans, various extracellular matrix components, hormones, growth factors, transforming factors, receptors or receptor complexes, natural polymers, synthetic polymers, DNA, RNA, olygonucleoytyides, a drug, a therapeutic agent, an anti-inflammatory agent, glycosaminoglicans, proteoglycans, morphogenic proteins glycoproteins, mucoproteins, mucopolysaccharides, matrix proteins, growth factors, transcription factors, anti-inflammatory agents, proteins, peptides, hormones, genetic material for gene therapy, a nucleic acid, a chemically modified nucleic acid, an oligonucleotide, ribonucleic acid, deoxyribonucleic acid, a chimeric DNA/RNA construct, DNA or RNA probes, anti-sense DNA, anti-sense RNA, a gene, a part of a gene, a composition including naturally or artificially produced oligonucleotides, a plasmid DNA, a cosmid DNA, viral and non-viral vectors required for promoting cellular uptake and transcription, a glycosaminoglycan, chondroitin 4-sulfate, chondroitin 6-sulfate, keratan sulfate, dermatan sulfate, heparin, heparan sulfate, hyaluronan, a lecitin rich interstitial proteoglycan, decorin, biglycan, fibromodulin, lumican, aggrecan, syndecans, beta-glycan, versican, centroglycan, serglycin, a fibronectin, fibroglycan, chondroadherins, fibulins, thrombospondin-5, an enzyme, an enzyme inhibitor, an antibody, and any combinations thereof.

Furthermore, in accordance with an embodiment of the invention, the reacting also includes adding one or more living cells to the at least one polysaccharide and the at least one reducing sugar before, during or after said cross-linking, to form a cross-linked matrix containing at least one live cell embedded in the matrix.

Furthermore, in accordance with an embodiment of the invention, the living cells are selected from vertebrate chondrocytes, osteoblasts, osteoklasts, vertebrate stem cells, embryonal stem cells, adult tissue derived stem cells, vertebrate progenitor cells, vertebrate fibroblasts, cells genetically engineered to secrete one or more of matrix proteins, glycosaminoglicans, proteoglycans, morphogenic proteins, growth factors, transcription factors, anti-inflammatory agents, proteins, hormones, peptides, one or more types of living cells engineered to express receptors to one or more molecules selected from the group consisting of proteins, peptides, hormones, glycosaminoglicans, proteoglycans, morphogenic proteins, growth factors, transcription factors, anti-inflammatory agents, glycoproteins, mucoproteins, and mucopolysaccharides, and any combinations thereof.

Furthermore, in accordance with an embodiment of the invention, the method further includes subjecting the cross-linked polysaccharide to a treatment selected from drying, freeze-drying, dehydration, critical point drying, molding, sterilization, homogenization, mechanical shearing, irradiation by ionizing radiation, irradiation by electromagnetic radiation, mixing with a pharmaceutically acceptable vehicle, impregnation with an additive and combinations thereof.

There is also provided, in accordance with an embodiment of the invention, a method for preparing cross-linked polysaccharides. The method includes the steps of reacting a polysaccharide with one or more reactants to form a derivatized form of the polysaccharide. The derivatized form contains one or more amino groups, and cross-linking the derivatized polysaccharide with at least one reducing sugar to form a cross-linked polysaccharide.

Furthermore, in accordance with an embodiment of the invention, the amino groups are selected from primary amino groups and secondary amino groups.

Furthermore, in accordance with an embodiment of the invention, the one or more reactants include a carbodiimide.

Furthermore, in accordance with an embodiment of the invention, the one or more reactants include a carbodiimide in the presence of adipic acid dihydrazide.

Furthermore, in accordance with an embodiment of the invention, the carbodiimide is 1-ethyl-3-(dimethyl aminopropyl)carbodiimide hydrochloride.

Furthermore, in accordance with an embodiment of the invention, the at least one reducing sugar is selected from an aldose, a ketose, and combinations thereof.

Furthermore, in accordance with an embodiment of the invention, the at least one reducing sugar is selected from glyceraldehyde, ribose, erythrose, arabinose, sorbose, fructose, glucose, D-ribose-5-phosphate, glucosamine, a diose, a triose, a tetrose, a pentose, a hexose, a septose, an octose, a nanose, a decose, glycerose, threose, erythrose, lyxose, xylose, arabinose, ribose, allose, altrose, glucose, fructose, mannose, gulose, idose, galactose, talose, a reducing monosaccharide, a reducing disaccharide, a reducing trisaccharide, a reducing oligosaccharide, derivatized forms of oligosaccharides, derivatized forms of monosaccharides, esters of monosaccharides, esters of oligosaccharides, salts of monosaccharides, salts of oligosaccharides, maltose, lactose, cellobiose, gentiobiose, melibiose, turanose, trehalose, isomaltose, laminaribiose, mannobiose and xylobiose, and combinations thereof.

There is also provided, in accordance with an embodiment of the invention, a method for preparing a composite cross-linked matrix. The method includes cross-linking with at least one reducing sugar at least one polysacharide selected from an amino-polysaccharide, an amino-functionalized polysaccharide and combinations thereof in the presence of at least one cross-linkable protein to form the composite cross-linked matrix.

Finally, there are also provided, in accordance with embodiments of the invention, cross-linked polysaccharides and composite matrices including polysaccharides and one or more proteins prepared by the methods described above.

### BRIEF DESCRIPTION OF THE DRAWINGS

In order to understand the invention and understand how it may be carried out in practice, several preferred embodiments will now be described, by way of non-limiting example only, with reference to the accompanying drawings:

FIG. 1 is a schematic graph representing the UV-Visible spectra of amino-functionalized Hyaluronic acid (AFHA) represented by the dashed curve and of DL-glyceraldehyde cross-linked AFHA represented by the solid curve obtained in accordance with an embodiment of the method of the present invention;

FIG. 2 is a schematic graph representing the UV-Visible spectra of D(−)-ribose cross-linked AFHA represented by the dashed curve, D(−)-erythrose cross-linked AFHA represented by the solid curve, and D(−)-arabinose cross-linked AFHA represented by the dotted curve, obtained in accordance with embodiments of the method of the present invention;

FIG. 3 is a schematic graph representing the UV-Visible spectra of non-cross-linked chitosan represented by the solid curve, D(−)-ribose cross-linked chitosan represented by the dashed curve, and DL-glyceraldehyde cross-linked chitosan represented by the dotted curve, obtained in accordance with embodiments of the method of the present invention;

FIG. 4 is a schematic graph representing the Fourier Transform Infrared (FTIR) spectra of hyaluronic acid represented by the dotted curve, AFHA represented by the dashed curve and DL-glyceraldehyde cross-linked AFHA represented by the solid curve, in accordance with embodiments of the method of the present invention;

FIGS. 5-7 are schematic graphs illustrating the results of measurements of the rheological properties of six different compositions of AFHA based polysaccharides cross linked with various different DL-glyceraldehyde concentrations for various times, in accordance with embodiments of the method of the present invention as compared to the rheological properties of some commercially available hyaluronic acid based matrices;

FIG. 8 is a schematic graph illustrating the results of measuring the swelling behavior of amino functionalized HA cross-linked with various different concentrations of DL-glyceraldehyde, in accordance with an embodiment of the present invention;

FIG. 9 is a schematic graph illustrating the results of measuring the swelling behavior of chitosan cross-linked with various different concentrations of DL-glyceraldehyde, in accordance with an embodiment of the present invention;

FIG. 10 is a schematic graph illustrating the carbazole assay results of digestion by hyaluronidase of DL-glyceraldehyde cross-linked amino-functionalized HA and commercially obtained Perlane®;

FIG. 11 is a schematic graph illustrating the carbazole assay results of FIG. 10 in which the absorbance values for Perlane® were multiplied by ten to compensate for the 10 fold dilution of the Perlane® test samples; and

FIG. 12 is a schematic graph illustrating the % resistance to hyaluronidase digestion in vitro of an exemplary sample of a D(−)-fructose cross-linked amino-functionalized HA matrix and of Perlane® as a function of digestion time.

### DETAILED DESCRIPTION OF THE INVENTION

### Notation Used Throughout

The following notation is used throughout this application.

| Term | Definition |
|---|---|
| μL | microliter |
| ADH | Adipic acid dihydrazide |
| AFHA | Amino functionalized Hyaluronic acid |
| AGE | Advanced glycation endproducts |
| CH | Chitosan |
| DI water | Deionized water |
| EDC | 1-ethyl-3-(dimethyl aminopropyl) carbodiimide |
| | hydrochloride |
| FTIR | Fourier Transform Infra-red |
| G | Gauge |
| HA | Hyaluronic acid |
| Hz | Hertz |
| IR | Infra-red |
| M | Molar |
| MDa | Million daltons |
| mg | milligram |
| mL | milliliter |
| mM | millimolar |
| Mw | Molecular weight |
| N | Normal |
| Pas | Pascal |
| PBS | Phosphate buffered saline |
| RPM | Revolutions per minute |

The present invention discloses a novel method for the preparation of novel cross-linked polysaccharide based biocompatible matrices and preparations having superior resistance to enzymatic degradation in-vivo and in-vitro, and other useful rheological and/or biological properties. The method is based, inter alia, on cross-linking amino-polysaccharides (such as, but not limited to chitosan) and/or amino-functionalized polysaccharides (such as, but not limited to, amino-functionalized hyaluronic acid) with reducing sugars such as D(−)-ribose, DL-glyceraldehyde, D(−)-erythrose, D(−)-arabinose and many other types of reducing sugars known in the art. Examples of such novel cross-linked matrices are also disclosed.

The present invention also discloses a novel method for the preparation of composite cross-linked matrices produced by cross-linking mixtures of one or more amino-polysaccharides and/or one or more amino-functionalized polysaccharides and/r one or more protein (and/or polypeptide) with one or more reducing sugar(s) (as the cross-linker) to form novel composite polysaccharide/protein based matrices and preparations having superior properties of resistance to enzymatic degradation in-vivo and in-vitro and other useful rheological and/or biological properties.

The terms “polysaccharide” and “polysaccharides” and their conjugate forms are used herein to define any naturally occurring and/or artificially prepared (and/or artificially synthesized) polysaccharide or polysaccharides, including any chemically modified forms and/or derivatives of such polysaccharide(s), and including, but not limited to esters and salts of such polysaccharides or of derivatized form thereof.

The terms “amino-polysaccharide” and “amino-polysaccharides” and their conjugate forms are used herein to define any form of polysaccharide or polysaccharides which contains one or more amino groups capable of being cross-linked by a reducing sugar.

The terms “amino-functionalized polysaccharide” and “amino-functionalized polysaccharides” and their conjugate forms are used herein to define any polysaccharide which has been chemically modified to attach thereto one or more chemical moieties including, inter alia, one or more amino groups which are capable of being cross-linked by a reducing sugar.

Thus, the cross-linking methods described herein may be used, inter alia, for cross-linking naturally occurring amino-polysaccharides, synthetic amino-polysaccharides, amino heteropolysaccharides, amino homopolysaccharides, amino-functionalized polysaccharides, hyaluronic acid and derivatized forms thereof, hyaluronan and derivatized forms thereof, chitosan and derivatized forms thereof, heparin and derivatized forms thereof, and various combinations thereof. The methods disclosed include the cross-linking of any suitable esters and salts of such amino-polysaccharides and amino-functionalized polysaccharides.

As will be appreciated by those skilled in carbohydrate chemistry, other types of amino group containing polysaccharides and/or amino functionalized polysaccharides which are not described in the specific examples and experiments hereinbelow, may also be cross-linked by the methods disclosed herein to provide a variety of cross-linked products. It is noted that the cross-linking of such amino polysaccharides or amino functionalized polysaccharides using reducing sugars (or reducing sugar derivatives) are included within the scope of the methods and products of the present invention.

Any suitable reducing sugar may be used as a cross-linking agent in the methods of present invention. The sugar may be a monosaccharide, a disaccharide having a reducing end, a trisaccharide having a reducing end, or the like. Suitable sugars may include aldoses and ketoses. When a monosaccharide is used as the cross-linker, it may be a triose, a tetrose, a pentose, a hexose, a heptose, but monosaccharides with more than seven carbon atoms may also be used. Thus, among the sugars that may be used in the novel cross-linking methods of the present invention are glycerose, threose, erythrose, lyxose, xylose, arabinose, allose, altose, glucose, manose, gulose, idose, galactose, fructose, talose, or any other diose, triose, tetrose, pentose, hexose, septose, octose, nanose, or decose and various suitable derivatized forms thereof.

Reducing derivatives of the above described monosaccharides or oligosaccharides which have an active aldehyde or keto group may also be used as the cross-linking agents in the present invention.

The rate of the cross-linking reaction may depend on the equilibrium concentration of the aldehyde or keto group existing in the open ring form of the particular sugar used, as is known in the art. However, it may be possible to compensate for the slow reaction rate of certain specific sugars by simply increasing the reaction time, as is known in the art.

The experiments described hereinbelow are non-limiting examples demonstrating typical reactions of such amino-polysaccharides and polysaccharides synthetically functionalized with amino groups with selected exemplary reducing sugars, and describing the improved degradation resistance and rheological properties of the resulting matrices. It is noted that the following experiments are given by way of example only and are not intended for limiting the scope of the present invention. Thus, as will be apparent to those skilled in the art, the polysaccharides, functionalized polysaccharides, reducing sugars (used as cross-linkers), reaction conditions, reaction mixture compositions, reaction temperatures, reaction duration, and the chemical, physical, rheological and biodurability properties of the resulting cross-linked matrices may vary from those particular described in the experiments disclosed hereinbelow.

The term hyaluronic acid (HA) is used in the following text as generic name to designate both hyaluronic acid per se and its salts or mixtures of salts and in particular salts of hyaluronate.

The term amino functionalized hyaluronic acid is used in the following text as generic name to designate hyaluronic acid and its salts or mixtures of salts which have been derivatized to contain moieties with a free amino group. The amino groups may be a primary amino groups and/or secondary amino groups. The preferred site for introducing the moiety containing the amino group is the carboxyl group of the polysaccharide but it may be possible to introduce such an amino group containing moiety at other sites on the saccharide ring(s). The amino-functionalization need not be full and some carboxyl groups (or other derivatization sites, if used) may remain non-derivatized.

The term amino functionalized polysaccharide is used in the following text as a generic term designating any polysaccharide which contains amino groups which may react with the aldehyde or keto group of the cross-linking reducing sugar. The amino groups may be primary and/or secondary amino groups. The amino groups may be positioned directly on the saccharide ring structure (as in chitosan) but may also be part of a moiety covalently linked to one or more sites or chemical groups on the sugar rings of the polysaccharide chain.

Thus, the amino group may be positioned directly on the sugar ring as in the case of chitosan (as disclosed in details hereinafter) which need not be functionalized and may be directly cross-linked with the reducing sugar through the ring backbone amino group. It is noted that partially de-acetylated chitin based polymers may also be cross-linked using the sugar cross-linking method of the present invention as may any polysaccharide having free amino groups (primary or secondary).

It is noted that in accordance with the results of the experiments disclosed herein the addition to the cross-linking reaction mixture of a polar, water miscible solvent such as, but not limited to ethanol may significantly increase the cross-linking efficiency and results in improved degradation resistance of the reaction products as compared to cross-linking in the presence of a buffered aqueous solution without any polar solvent.

While the exact reaction mechanisms of the cross-linking and the chemical nature of the resulting cross-linked polysaccharides is not presently fully understood, it is assumed that the reactions may be somewhat similar (though not necessarily identical) to the classical glycation reaction in which a reducing sugar is used to cross-link protein molecules based on a reaction of the sugar's aldehyde or keto group with amino groups of amino acids of the proteins, such as, for example with the free amino group of a lysine or arginine or other amino acids present in the protein's chain.

These protein cross-linking reactions of reducing sugars are well known in the art. It is believed that such protein cross-linking reaction proceeds at least partially through Amadori rearrangement of the initial reaction product leading to the formation of yellowish or brown advanced glycation products.

While the inventors of the present invention have not fully characterized the structure of the cross-linked polysaccharides obtained in the experiments disclosed the present application, the characteristic absorbance peaks in the range of 225-235, and 285-355 nanometers of the resulting cross-linked polysaccharides may be an indication of the presence of glycation products of somewhat similar (but not necessarily identical) nature to protein glycation products and advanced protein glycation products (AGE).

Materials Used in Experiments

Heparin sodium EP (Batch No. 9818030) was obtained from JUK Kraeber GmbH & Co, Hamburg, Germany.

Restylane® (Lot number 7349) and Restylane-Perlane (Lot Number 7064) is commercially available from Q-Med AB, Uppsala Sweden. Hylaform® Plus (Lot number R0409 is commercially available from Genzyme Biosurgery (a division of Genzyme Corporation) through their distributor INAMED AESTHETICS, Ireland.

Turrax homogenizations were performed using a model ULTRA TURRAX® T-25 (basic), commercially available from, IKA®-WERKE, Germany, unless otherwise stated.

All lyophilization procedures were carried out using a model FD 8 Freeze dryer commercially available from Heto Lab Equipment, Denmark. The condenser temperature was −80° C. The shelf temperature during pre-freezing was −40° C. The shelf temperature for lyophilization was +30° C. The pre-freezing time was 8 hours and the lyophilization time was 24 hours. The vacuum during lyophilization was approximately 0.01 bar.

TABLE 1 below lists the commercial sources of materials used in experiments described in the present application

TABLE 1

| Material | Supplier | Cat. No. |
|---|---|---|
| N-Ethyl-N′-(3- | Sigma | E7750 |
| dimethylaminopropyl)carbodiimide | | |
| Hydrochloride | | |
| Adipic acid dihydrazide | Sigma | A0638 |
| Chitosan, medium molecular weight | Aldrich | 448877 |
| D(−)-Arabinose | Sigma | A3131 |
| D(−)-Erythrose | Fluka | 18934 |
| DL-Glyceraldehyde | Sigma | G5001 |
| Cytochrome C from bovine heart | Sigma | C2037 |
| D-Ribose-5-phosphate Disodium salt | Sigma | 83875 |
| dihydrate | | |
| D(−) Fructose | Fluka | F0127 |
| D(−) Ribose | Sigma | R7500 |
| D(+) Glucose | Sigma | 49159 |
| D(+) Sorbose | Sigma | S4887 |
| L(−) Sorbose | Sigma | S3695 |
| L(+) Fructose | Sigma | 31140 |
| D(+) Glucosamine hydrochloride | Sigma | G4875 |
| Maltose Monohydrate | Sigma | M5885 |
| D(+) Lactose Monohydrate | Sigma | 61340 |
| 1-Butanol | Fluka | 19430 |
| 1-Hexanol | Fluka | 52840 |
| 2-Propanol | Aldrich | 32,047-1 |
| Acetone | Fluka | 00585 |
| Ethyl acetate | Fluka | 45770 |
| Dichloromethane | Sigma-Aldrich | 443484 |
| Diethyl ether | Riedel de Haen | 32203 |
| Ethanol (absolute) | Merck | 100983 |
| Hexane | Fluka | 52770 |
| Toluene | Fluka | 89682 |

HA Amino Functionalization Procedure I

400 mg of HA 150 (commercially available as product No. 2222003, Sodium Hyaluronate Pharma Grade 150 from NovaMatrix FMC Biopolymer, Oslo, Norway) having a molecular weight in the range of 1.4-1.8 MDa, or of HA 80 (commercially available as product No. 2222002, Sodium Hyaluronate Pharma Grade 150 from NovaMatrix FMC Biopolymer, Oslo, Norway) having a molecular weight in the range of 0.62-1.15 MDa were dissolved in 350 mL of DI water, 7 grams of adipic acid dihydrazide (ADH) were added to the mixture. The pH of the resulting solution was adjusted to 4.75 and the solution was stirred for two hours. 764 mg of 1-ethyl-3-(dimethyl aminopropyl)carbodiimide hydrochloride was dissolved in 2.0 mL of DI water and added to the mixture and the pH was again adjusted to 4.75 at room temperature. The reaction was monitored by the change of the pH and continuously adjusted to 4.75. After no change of pH could be detected, the reaction mixture was left for an additional hour or over night. Subsequently, the solution was transferred into a dialysis tube and dialyzed against DI water until no ADH was detected in the dialyzate. The dialyzed solution was transferred into 3.5 liters of 100% ethanol, 2 grams of NaCl were added and the mixture was stirred for one hour. In order to separate the precipitated modified HA, the solution was centrifuged for 20 minutes at 7000 rpm and the supernatant was removed. The amino-functionalized HA (AFHA) obtained was stored at 4° C. until used.

It is noted that in all the cross-linking experiments using AFHA described below, the amino functionalized HA resulting from functionalizing HA 80 is consistently referred to as AFHA80 hereinafter, and the amino functionalized HA resulting from functionalizing HA 150 is consistently referred to as AFHA I 150, hereinafter. This two amino-functionalized HA materials (AFHA80 and AFHA I 150) were used for all the HA cross-linking experiments disclosed hereinbelow).

HA Cross-Linking Procedures

In all the experiments described hereinafter vortexing was performed using a vortex™ rotary mixer. All centrifugations (unless specifically stated otherwise) were done using a model RC5C centrifuge with a SORVALL SS-34 rotor commercially available from SORVALL® Instruments DU PUNT, USA.

Each of the following experiments described below were performed such that the first number (referring to the experimental series number) is followed by a slash (/) sign and then by the range of the actual experiments performed within the series. For example, EXPERIMENT SERIES 32/1-3 below includes the following three experiments: Experiment 32/1, Experiment 32/2, and Experiment 32/3. This notation is consistently used throughout the specification.

### Experiment Series 32/1-3

Approximately 5 mg AFHA80 was dissolved in 1 mL of DI water and added to 5 mL 100% ethanol and vortexed for 1 minute, after which the following different amounts of glyceraldehyde were added to the AFHA80 mixture as follows:

a) 2 mg of glyceraldehyde dissolved in 100 μL of DI water (Experiment 32/1)

b) 4 mg of glyceraldehyde dissolved in 200 μL of DI water (Experiment 32/2)

c) 6 mg of glyceraldehyde dissolved in 300 μL of DI water (Experiment 32/3)

The resulting reaction mixture was vortexed for 1 minute and placed into an incubator and rotated for 24 hours at 37° C. Afterwards, the solution was centrifuged at 6000 rpm for 20 minutes, the supernatant was removed and 1 mL of DI water was added to the remaining pellet. After 30 minutes at room temperature the mixture was centrifuged again at 6000 rpm for 20 minutes. The resulting cross-linked products had the following characteristics:

a) (Experiment 32/1): 500 μL of hard gel.

b) (Experiment 32/2): A soft, opaque gel with no phase separation between cross-linked HA and water (which after 3 hours of re-centrifugation resulted in 500 μL of clear gel).

c) (Experiment 32/2): 800 μL of gel.

### Experiment 33/1

Approximately 25 mg of AFHA80 were dissolved in 5 mL of DI water, added to 25 mL of 100% ethanol and vortexed for 1 minute. A solution of 10 mg of DL-glyceraldehyde dissolved in 500 μL of DI water was added to the mixture and the resulting mixture was vortexed for 1 minute, placed into an incubator and rotated for 24 hours at 37° C. After 6 hours of rotating in the incubator an additional 5 mg glyceraldehyde dissolved in 250 μL of DI water was added to the reaction mixture and the mixture was returned to the incubator to complete the incubation period. At the end of the 24 hour incubation, the solution was centrifuged at 6000 rpm for 20 minutes, the supernatant was removed and 40 mL of DI water and 2 mL of PBS buffer (10 mM) were added to the pellet and left at room temperature for 6 hours. The mixture was then centrifuged again at 6000 rpm for 20 minutes. The resulting product was 500 μL of a hard, opaque gel.

### Experiment Series 35/1-4

Approximately 5 mg of AFHA80 were dissolved in 1 mL of DI water and added to 12 mL of 100% ethanol and vortexed for 1 minute, after which the following different amounts of DL-glyceraldehyde were added as follows:

a) 1.5 mg of DL-glyceraldehyde dissolved in 75 μL DI water (Experiment 35/1).

b) 3.0 mg of DL-glyceraldehyde dissolved in 150 μL DI water (Experiment 35/2).

c) 4.5 mg of DL-glyceraldehyde dissolved in 225 μL DI water (Experiment 35/3).

d) 6.0 mg of DL-glyceraldehyde dissolved in 300 μL DI water (Experiment 35/4).

The resulting reaction mixtures were vortexed for 1 minute, placed into an incubator and rotated for 24 hours at 37° C. After 24 hours, the solutions were centrifuged at 6000 rpm for 20 minutes, the supernatant was removed and 5 mL of DI water were added to each pellet. After 30 minutes at room temperature the mixtures were centrifuged again at 6000 rpm for 20 minutes. The resulting cross-linked products had the following characteristics:

a) (Experiment 35/1) 3.4 mL of transparent gel.

b) (Experiment 35/2) 3.5 mL of transparent gel.

c) (Experiment 35/3) 4.0 mL of transparent gel.

d) (Experiment 35/4) 4.0 mL of transparent gel.

### Experiment Series 37/4-6

Approximately 5 mg of AFHA80 were dissolved in 1 mL of DI water and added to 10 mL of 100% ethanol. The mixture was vortexed for 1 minute, after which the following different amounts of DL-glyceraldehyde were added to the mixture as follows:

a) 8 mg of DL-glyceraldehyde dissolved in 400 μL DI water (Experiment 37/4).

b) 10 mg of DL-glyceraldehyde dissolved in 500 μL DI water (Experiment 37/5).

c) 12 mg of DL-glyceraldehyde dissolved in 600 μL DI water (Experiment 37/6).

The resulting reaction mixtures were vortexed for 1 minute and placed into an incubator and rotated for 24 hours at 37° C. At the end of the incubation period the solutions were centrifuged at 6000 rpm for 20 minutes, the supernatant was removed and 5 mL DI water added to each pellet. After 30 min at room temperature the mixtures were centrifuged again at 6000 rpm for 20 minutes. The resulting cross-linked products had the following characteristics:
a) (Experiment 37/4) 2.5 mL of transparent gel.
b) (Experiment 37/5) 1.9 mL of transparent gel.
c) (Experiment 37/6) 1.5 mL of transparent gel.

### Experiment Series 38/1-3

Approximately 5 mg of AFHA80 were dissolved in 1 mL of DI water and added to 10 mL of 100% ethanol and vortexed for 1 minute, after which the following different amounts of DL-glyceraldehyde were added to the mixture as follows:

a) 14 mg of DL-glyceraldehyde dissolved in 700 μL DI water (Experiment 38/1).

b) 16 mg of DL-glyceraldehyde dissolved in 800 μL DI water (Experiment 38/2).

c) 18 mg of DL-glyceraldehyde dissolved in 900 μL DI water (Experiment 38/3).

The resulting reaction mixtures were vortexed for 1 minute, placed into an incubator and rotated for 24 hours at 37° C. After 24 hours, the solutions were centrifuged at 6000 rpm for 20 minutes, the supernatant was removed and 5 mL of DI water were added to each of the pellets. After 30 minutes at 37° C. the mixtures were centrifuged again at 6000 rpm for 20 minutes. The resulting cross-linked products had the following characteristics:

a) (Experiment 38/1) 1.0 mL of transparent gel.

b) (Experiment 38/1) 0.75 mL of transparent gel.

c) (Experiment 38/1) 0.50 mL of transparent gel.

### Experiment Series 41/1-4

Approximately 5 mg of AFHA I 150 were dissolved in 5 mL of DI water, added to 10 mL 100% ethanol and vortexed for 1 minute, after which the following different amounts of DL-glyceraldehyde were added to the mixture as follows:

a) 6 mg of DL-glyceraldehyde dissolved in 300 μL DI water (Experiment 41/1).

b) 8 mg of DL-glyceraldehyde dissolved in 400 μL DI water (Experiment 41/2).

c) 10 mg of DL-glyceraldehyde dissolved in 500 μL DI water (Experiment 41/3).

d) 12 mg of DL-glyceraldehyde dissolved in 600 μL DI water (Experiment 41/4).

The resulting reaction mixtures were vortexed for one minute, placed into an incubator and rotated for 24 hours at 37° C. After 24 hours, the solutions were centrifuged at 6000 rpm for 20 minutes, the supernatant was removed and 20 mL of DI water were added to each of the pellets. After 30 minutes at 37° C. the mixtures were centrifuged again at 6000 rpm for 20 minutes. No phase separation was observed in cases a (Experiment 41/1), b (Experiment 41/2) and c (Experiment 41/3); 5 mL supernatant could be removed in case d (Experiment 41/4). 50 mL of 1N NaOH solution were then added to all the samples, and the samples were again centrifuged and washed twice with 40 mL of PBS buffer (10 mM, pH 7.36). The diluted cross-linked products were centrifuged at 6000 rpm for 20 min after each step and the supernatant was removed. The results were as follows: in samples a (Experiment 41/1), b (Experiment 41/2) and c (Experiment 41/3), no gel remained. In sample d (Experiment 41/4), 5 mL of transparent gel were obtained.

### Experiment Series 42/1-3

Approximately 5 mg of AFHA I 150 were dissolved in 5 mL of DI water, added to 40 mL of 100% ethanol and vortexed for 1 min, after which the following different amounts of DL-glyceraldehyde were added to the mixture as follows:

a) 16 mg of DL-glyceraldehyde dissolved in 800 μL DI water (Experiment 42/1).

b) 20 mg of DL-glyceraldehyde dissolved in 1000 μL DI water (Experiment 42/2).

c) 40 mg of DL-glyceraldehyde dissolved in 2000 μL DI water (Experiment 42/3).

The resulting reaction mixtures were vortexed for 1 minute and placed into an incubator and rotated for 24 hours at 37° C. After 24 hours, the solutions were centrifuged at 6000 rpm for 20 minutes, the supernatants were removed and 40 mL of DI water were added to each of the resulting pellets. After 30 minutes at room temperature, each of the mixtures was centrifuged at 6000 rpm for 20 minutes. The resulting cross-linked gels exhibited increasing gel viscosity from a) through b) to c) (i.e the gel resulting in Experiment 42/1 had the lowest viscosity of the three, the gel resulting in Experiment 42/3 had the highest viscosity of the three and the resulting in Experiment 42/2 had a viscosity value between the highest and the lowest values of the three samples).

### Experiment Series 44/1-2

Approximately 5 mg of AFHA80 were dissolved in 5 mL of DI water, added to 40 mL of 100% ethanol and vortexed for 1 min, after which the following different amounts of different reducing sugar cross-linkers were added to the mixture as follows:

a) 44 mg DL-glyceraldehyde dissolved in 2 mL of DI water (Experiment 44/1).

b) 44 mg D(−)-ribose dissolved in 2 mL of DI water (Experiment 44/1).

The reaction mixtures were vortexed for 1 minute, placed into an incubator and rotated for 24 hours at 37° C. (Experiment 44/1), and for eleven (11) days at 37° C. (Experiment 44/2). At the end of the incubation period each of the reaction mixtures, was centrifuged at 6000 rpm for 20 min, the supernatant was removed and 40 mL of physiologic NaCl solution (0.9%) was added to the each of the resulting pellets. The mixtures were left for 30 minutes at room temperature and centrifuged at 6000 rpm for 20 minute. The results were as follows: a) (Experiment 44/1) a soft transparent gel was obtained b) (Experiment 44/2) an off white to yellowish gel was obtained.

### Experiment Series 53/1-3

Approximately 50 mg of AFHA I 150 were dissolved in 2 mL of DI water. 100 mg of DL-glyceraldehyde were dissolved in 2 mL of DI water. The two solutions were mixed and extruded five times through a syringe without a needle and twice through a 18G needle. The mixture was finally extruded through a 18G needle into the following quantities of ethanol:

a) 20 mL of 100% ethanol (Experiment 53/1).

b) 30 mL of 100% ethanol (Experiment 53/2).

c) 40 mL of 100% ethanol (Experiment 53/3).

Each of the resulting cross-linking reaction mixtures was placed into an incubator and rotated for 24 hours at 37° C. At the end of the incubation period, each of the solutions was centrifuged at 6000 rpm for 20 minutes. The supernatants were removed and 20 mL of physiologic NaCl solution (0.9%) was added to each of the resulting pellets. The mixtures were then left for three hours at 37° C. and were centrifuged at 6000 rpm for 20 minutes.

### Experiment 54/1

Approximately 50 mg of AFHA I 150 were dissolved in 4 mL of DI water, containing 150 mg DL-glyceraldehyde. The mixture was repeatedly extruded through a 22 G needle (six times) and then extruded through a 18G needle into 40 mL of 100% ethanol, placed into an incubator and rotated for 24 hours at 37° C. After incubation, the solution was centrifuged at 6000 rpm for 20 minutes, the supernatant was removed and 20 mL of physiologic NaCl solution (0.9%) were added to the pellet. The resulting mixture was left for two hours at 37° C. and the mixture was then centrifuged at 6000 rpm for 20 minutes.

### Experiment 55/1

Approximately 50 mg of AFHA I 150 were dissolved in 4 mL of DI water, containing 150 mg DL-glyceraldehyde. The mixture was repeatedly extruded through a 22 G needle (six times) and then extruded through a 18G needle into a mixture of 35 mL 100% of ethanol and 5 mL of DI water. The reaction mixture was placed into an incubator and rotated for 24 hours at 37° C. After the incubation period, the mixture was centrifuged at 6000 rpm for 20 minutes, the supernatant was removed and 20 mL of physiologic NaCl solution (0.9%) were added to the pellet. After two hours at 37° C., the mixture was centrifuged at 6000 rpm for 20 minutes. The resulting white material, showed no water uptake.

### Experiment 60/1

Approximately 50 mg of AFHA I 150 were dissolved in 4 mL of DI water containing 300 mg DL-glyceraldehyde and shaken for 30 minutes at 50° C. The reaction mixture was then extruded through a syringe (without a needle) into 40 mL of 100% ethanol and the resulting mixture was placed into a water bath and shaken for six hours at 50° C. The mixture was then centrifuged at 6000 rpm for 20 min, the supernatant was removed and 40 mL of physiologic NaCl solution (0.9%) together with 2 mL of PBS buffer solution (10 mM, pH 7.36) were added to the pellet. The mixture was then centrifuged at 6000 rpm for 20 minutes. The resulting reaction product was 2.8 mL of gel.

### Experiment 61/1

Approximately 50 mg of AFHA I 150 were dissolved in 4 mL of DI water containing 300 mg DL-glyceraldehyde and the mixture was shaken for 60 minutes at 50° C. The mixture was then extruded through a syringe (without a needle) into 40 mL of 100% ethanol and the resulting mixture was placed into a water bath and shaken for five hours at 50° C. After the incubation, the mixture was centrifuged at 6000 rpm for 20 minutes, the supernatant was removed and 40 mL of physiologic NaCl solution (0.9%) together with 2 mL of PBS buffer solution (10 mM, pH 7.36) were added to the pellet. The mixture was then centrifuged at 6000 rpm for 20 minutes.

### Experiment 62/1

Approximately 50 mg of AFHA I 150 were dissolved in 4 mL of DI water, containing 300 mg DL-glyceraldehyde and shaken for 10 minutes at room temperature. The mixture was released through a syringe into 40 mL 100% ethanol, placed into a water bath and shaken for 24 hours at 50° C. Afterwards, the solution was centrifuged at 6000 rpm for 20 minutes, the supernatant was removed and 40 mL of physiologic NaCl solution (0.9%) together with 2 mL of PBS buffer solution (10 mM, pH 7.36) was added to the pellet. The mixture was then centrifuged at 6000 rpm for 20 minutes. The resulting product was 1 mL of opaque gel.

### Experiment Series 65/3-5

Approximately 50 mg of AFHA I 150 were dissolved in 4 mL of DI water, containing:

a) 100 mg DL-glyceraldehyde (Experiment 65/3).

b) 200 mg DL-glyceraldehyde (Experiment 65/4).

c) 300 mg DL-glyceraldehyde (Experiment 65/5).

The resulting reaction mixtures were extruded four times through a 20G needle and each of the reaction mixtures was extruded through a syringe without a needle into 40 mL of 100% ethanol and placed into a heating bath and shaken for three (3) hours at 50° C. and then placed into an incubator and rotated for sixteen (16) hours at 37° C. Each of the resulting reaction mixtures was then centrifuged at 6000 rpm for 20 minutes, the supernatants were removed and 40 mL of physiologic NaCl solution (0.9%) together with 2 mL of PBS buffer solution (10 mM, pH 7.36) was added to each of the resulting pellets. The mixtures were then centrifuged again at 6000 rpm for 20 minutes. The resulting reaction products had the following appearance:

a) (Experiment 65/3) 3.5 mL of opaque gel.

b) (Experiment 65/3) 2.5 mL of opaque gel.

c) (Experiment 65/3) 2.0 mL of opaque gel.

### Experiment Series 67/1-2

Approximately 50 mg of AFHA I 150 were dissolved in 4 mL of DI water containing 50 mg DL-glyceraldehyde. The material was mixed in a syringe and released into 40 mL 100% ethanol. The mixture was placed into an incubator and rotated for the following periods:
a) 2 days at 37° C. (Experiment 67/1).
b) 3 days at 37° C. (Experiment 67/2).

After the incubation period the solutions were centrifuged at 9000 rpm for 20 minutes, the supernatant was removed and 40 mL of physiologic NaCl solution (0.9%) together with 2 mL of PBS buffer solution (10 mM, pH 7.36) were added to each of the pellets. The mixtures were then centrifuged again at 9000 rpm for 20 minutes. The resulting reaction products were: a) 2 mL of opaque gel (Experiment 67/1), and b) 1.6 mL of opaque gel (Experiment 67/2).

### Experiment Series 67/4-6

Approximately 50 mg of AFHA I 150 were dissolved in 4 mL of DI water, containing:

a) 300 mg D(−)-ribose (Experiment 67/4).

b) 300 mg D(−)-arabinose (Experiment 67/5).

c) Approximately 150 mg D(−)-erythrose (Experiment 67/6).

The three mixtures were each mixed by extruding for times through a 20G needle in a syringe and then each extruded through the 20G needle into 40 mL of 100% ethanol. The mixtures were then placed into an incubator and rotated for 15 days at 37° C. After the incubation, the mixtures were centrifuged at 9000 rpm for 20 minutes, the was supernatant removed, and 40 mL of physiologic NaCl solution (0.9%) together with 2 mL of PBS buffer solution (10 mM, pH 7.36) were added to the pellets and the mixture were centrifuged again at 9000 rpm for 20 minutes. The resulting reaction products showed the properties as follows:

a) (Experiment 67/4) No water uptake—yellowish fibers

b) (Experiment 67/5) Transparent gel.

c) (Experiment 67/6) No water uptake—white fibers

### Experiment 72/1

Approximately 100 mg of AFHA I 150 were dissolved in 4 mL of DI water, containing 100 mg DL-glyceraldehyde. The resulting reaction mixture was extruded four times through an 18G needle and then extruded twice through a 21G needle. The mixture was split into two equal portions. Each of the two portions was extruded through the 21G needle into 40 mL of 100% ethanol. The resulting mixtures were placed into an incubator and rotated for three days at 37° C. After the incubation period, 5 mL of physiological NaCl solution (0.9%) was added to each of the two mixtures and the solution was centrifuged at 9000 rpm for 20 minutes. The supernatants were removed and 40 mL of physiological NaCl solution (0.9%) together with 2 mL of PBS buffer solution (10 mM, pH 7.36) were added to each of the resulting pellets. The mixtures were then centrifuged at 9000 rpm for 20 minutes and the resulting pellets were combined. The experiment resulted in a total (combined) volume of 2.1 mL of opaque gel.

### Experiment Series 75/1, 2

100 mg of DL-glyceraldehyde were dissolved in 7 mL of DI water. A slurry of 100 mg of AFHA I 150 in 1.5 mL of 100% ethanol was added to the prepared DL-glyceraldehyde solution. The resulting mixture was homogenized by extrusion (three times) through an 18G needle and split into two equal portions. Each of the two portions was extruded into 40 mL of 100% ethanol. The mixtures were then placed into an incubator and rotated for 3 days at 37° C. Afterwards, 5 mL physiological NaCl solution (0.9%) were added to each of the two mixtures and the solutions were centrifuged at 9000 rpm for 20 minutes. The supernatants were removed and 40 mL physiological NaCl solution (0.9%) together with 2 mL PBS buffer solution (10 mM, pH 7.36) were added to each of the resulting pellets. The two mixtures were then centrifuged again at 9000 rpm for 20 minutes and the two pellets were combined.

### Experiment Series 75/3, 4

80 mg of DL-glyceraldehyde were dissolved in 7 mL of DI water. A slurry of 100 mg of AFHA I 150 in 2 mL of 100% ethanol was added in the prepared DL-glyceraldehyde solution. The resulting mixture was homogenized by extrusion (three times) through an 18G needle and split into two equal portions. Each of the two portions was separately extruded into 40 mL of 100% ethanol. The mixtures were then placed into an incubator and rotated for 3 days at 37° C. Afterwards, 5 mL of physiological NaCl solution (0.9%) were added to each of the two reaction mixtures and the mixtures were centrifuged at 9000 rpm for 20 minutes. The supernatants were removed and 40 mL of physiological NaCl solution (0.9%) together with 2 mL of PBS buffer solution (10 mM, pH 7.36) were added to each of the resulting pellets. The pellets were then centrifuged again at 9000 rpm for 20 minutes and the resulting pellets were combined.

### Experiment 77/1

90 mg of DL-glyceraldehyde were dissolved in 14 mL of DI water. A slurry of 100 mg of AFHA I 150 in 5 mL of 100% ethanol was added to the prepared DL-glyceraldehyde solution. The resulting reaction mixture was homogenized by extrusion (three times) through an 18G needle and split into two equal portions. Each of the portions was added into 40 mL of 100% ethanol. The resulting mixtures were placed into an incubator and rotated for 3 days at 37° C. Afterwards, 5 mL of physiological NaCl solution (0.9%) were added to each of the mixtures and the mixtures were centrifuged at 9000 rpm for 20 minutes. The supernatants were removed and 40 mL of physiological NaCl solution (0.9%) together with 2 mL of PBS buffer solution (10 mM, pH 7.36) were added to each of the resulting pellets and mixed. The mixtures were centrifuged again at 9000 rpm for 20 minutes and the resulting pellets were combined.

Chitosan Cross-Linking Procedures:

The fibrillation buffer used in the experiments was prepared as follows as follows: 6.5 Liters DI water were placed into a 10 Liter glass vessel. 11.3 gram of NaOH (for a 0.04M final concentration) and 252 gram of Na₂HPO₄.2H₂O (for a 0.2M final concentration) were dissolved in the DI water. The pH was adjusted to 11.2 with 10N NaOH. The volume of the solution was completed to 7 Liters with DI water. The final pH was adjusted (with NaOH) to the range of pH 11.20-11.30.

### Experiment 9/1

Approximately 181.5 mg of chitosan were dissolved in 9.6 mL of 0.1N HCl. 14 mg of DL-glyceraldehyde were dissolved in 2.5 mL of DI water and mixed with the chitosan solution. The mixture was vortexed for 1 minute and 1 mL of fibrillation buffer and 9.6 mL of 100% ethanol were slowly added to the chitosan/DL-glyceraldehyde mixture under constant stirring. The reaction mixture was placed in an incubator and rotated for 24 hours at 37° C. At the end of the incubation a solution containing 28 mg DL-glyceraldehyde dissolved in 1.4 mL of DI water was added to the mixture and the resulting mixture was left in an incubator and rotated for an additional 24 hours at 37° C. The mixture was then centrifuged at 7000 rpm for 15 minutes, the supernatant was removed and the resulting pellet was washed with 30 mL of 1N HCl and then with 30 mL of DI water. Each washing step included a centrifugation of the sample in order to remove the excess of liquid. The Resulting pellet was of a firm gel consistency.

### Experiment 12/1

Six different solutions of glyceraldehyde were prepared as follows:

a) 20 mg of DL-glyceraldehyde in 2.5 mL of DI water.

b) 40 mg of DL-glyceraldehyde in 2.5 mL of DI water.

c) 60 mg of DL-glyceraldehyde in 2.5 mL of DI water.

d) 80 mg of DL-glyceraldehyde in 2.5 mL of DI water.

e) 100 mg of DL-glyceraldehyde in 2.5 mL of DI water.

Each of the resulting DL-glyceraldehyde solutions a-e was separately mixed with a solution prepared by dissolving 196 mg chitosan 10 mL of 0.1N HCl. Each of the resulting six reaction mixtures was vortexed for 1 minute. To each of the six mixtures 1 mL of fibrillation buffer was added slowly under constant stirring followed by 15 mL of 70% ethanol/DI water mixture (v/v) which were also slowly added under constant stirring. The reaction mixture was then placed in an incubator and rotated for 24 hours at 37° C. After the 24 hour incubation 5 mL PBS buffer and 2.5 mL fibrillation buffer were slowly added to the reaction mixture followed by vortexing. The mixtures were then re-incubated at 37° C. During the second incubation, the mixture was taken out of the incubator twice, vortexed and then returned to incubator (one and two hours after adding the PBS and fibrillation buffers). The mixtures were than left in the incubator and rotated. The total incubation time at 37° C. was 48 hours. After completion of the incubation, the six samples (a-e) were centrifuged at 7000 rpm for 15 minutes. The supernatants were removed and the product was washed with 30 mL of 1N HCl followed by 30 mL of DI water. Each washing step included a centrifugation of the sample in order to remove the excess of solvent. All the five resulting samples exhibited a yellowish color (which was stronger than the initial yellowish color of the unreacted chitosan solution) probably due to the formation of a glycation product. A clear cut phase separation resulting in an observed gel phase was only found in samples a and b.

### Experiment Series 35/1-4

The following four different DL-glyceraldehyde solutions were prepared:

a) 15 mg of DL-glyceraldehyde dissolved in 75 μL of PBS (Experiment 35/1).

b) 30 mg of DL-glyceraldehyde dissolved in 150 μL of PBS (Experiment 35/2).

c) 45 mg of DL-glyceraldehyde dissolved in 225 μL of PBS (Experiment 35/3).

d) 60 mg of DL-glyceraldehyde dissolved in 300 μL of PBS (Experiment 35/4).

Each of the above four DL-glyceraldehyde solutions a-d was separately mixed with a solution of approximately 20 mg chitosan dissolved in 1 mL of 0.1N HCl and neutralized (to pH 7.0), using 0.1N HCl. Each of the resulting four reaction mixtures was vortexed for 1 minute and 12 mL of 100% ethanol were added to each vortexed reaction mixture under constant stirring. The resulting reaction mixtures were then placed in an incubator and rotated for 24 hours at 37° C. After the completion of the incubation period, the reaction mixtures were centrifuged at 7000 rpm for 15 minutes. The supernatants were removed and the resulting pellets were each washed with 10 mL of 1N HCl followed by washing with 5 mL of DI water. Each washing step included a centrifugation of the sample in order to remove the excess of solvent. No phase separation between water and the chitosan gel could be observed in any of the four samples a-d (of experiments 37/1, 37/2, 37/3 and 37/4, respectively).

### Experiment Series 38/1-6, and 39/1-4

Approximately 20 mg of chitosan were dissolved in 1 mL of 0.1N HCl and neutralized, using 0.1 N NaCl. Ten different solutions of DL-glyceraldehyde were prepared as follows:
a) 80 mg glyceraldehyde were dissolved in 400 μL PBS (Experiment 38/1).
b) 100 mg of DL-glyceraldehyde were dissolved in 500 μL of PBS (Experiment 38/2).
c) 120 mg of DL-glyceraldehyde were dissolved in 600 μL of PBS. (Experiment 38/3).
d) 160 mg of DL-glyceraldehyde were dissolved in 800 μL of PBS (Experiment 38/4).
e) 200 mg of DL-glyceraldehyde were dissolved in 1000 μL of PBS (Experiment 38/5).
f) 240 mg of DL-glyceraldehyde were dissolved in 1200 μL of PBS (Experiment 38/6).
g) 300 mg of DL-glyceraldehyde were dissolved in 1500 μL of PBS (Experiment 39/1).
h) 350 mg of DL-glyceraldehyde were dissolved in 1750 μL of PBS (Experiment 39/2).
i) 400 mg of DL-glyceraldehyde were dissolved in 2000 μL of PBS (Experiment 39/3).
j) 500 mg of DL-glyceraldehyde were dissolved in 2500 μL of PBS (Experiment 39/4).

Each of the DL-glyceraldehyde solutions a-j was then separately mixed with I mL of a chitosan solution containing approximately 20 mg chitosan dissolved in 1 mL of 0.1N HCl and neutralized (to pH 7.0), using 0.1N HCl. Each reaction mixture was vortexed for 1 minute and 10 mL of 100% ethanol were added to each of the reaction mixtures under constant stirring. The reaction mixtures were then placed in an incubator and rotated for 24 hours at 37° C. After the completion of the incubation period, the reaction mixtures were centrifuged at 7000 rpm for 15 minutes, the supernatants were removed and the resulting pellets were each washed with 10 mL of 1 N HCl followed by washing with 5 mL of DI water (for experiments 38/1-6 above) or with 10 mL of DI water (for experiments 39/1-4 above). Each washing step included a centrifugation of the sample in order to remove the excess of solvent. In the final reaction products of experiments 38/1-6, no phase separation between water and the cross-linked chitosan gel could be observed. In the final reaction products of 39/1-4, phase separation into a supernatant and a chitosan gel was observed after centrifugation. The color of the reaction products of experiments 38/1-6 and 39/1-4 was observed to vary from off-white to yellowish, with concomitant decreasing water uptake of the resulting cross-linked chitosan gel for increasing cross-linker (DL-glyceraldehyde) concentrations.

### Experiment Series 40/1-3

In this experiments the conditions were an exact repetition of Experiment 39 described above except that only parts g, i and j were performed with DL-glyceraldehyde concentrations as follows:

g) 300 mg of DL-glyceraldehyde were dissolved in 1500 μL of PBS (Experiment 40/1).

i) 400 mg of DL-glyceraldehyde were dissolved in 2000 μL of PBS (Experiment 40/2).

j) 500 mg of DL-glyceraldehyde were dissolved in 2500 μL of PBS (Experiment 40/3).

The rest of the reaction conditions were performed exactly as in EXPERIMENT SERIES 39/1-4, as described in detail hereinabove.

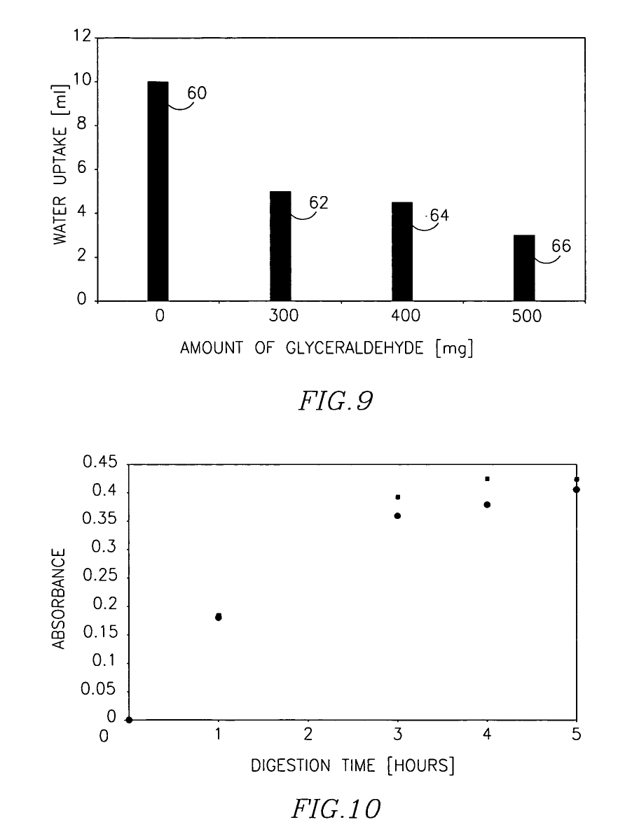

The resulting pellets were used for measuring the swelling behavior of the products (the results are illustrated in FIG. 9).

### Experiment Series 44/3-4

a) 56 mg of DL-glyceraldehyde were dissolved in 500 μL of DI water (Experiment 44/3).
b) 56 mg of D(−)-ribose were dissolved in 500 μL of DI water (Experiment 44/4).

Each of the solutions of a and b above was separately mixed with a chitosan solution prepared by dissolving approximately 100 mg chitosan in 5 mL of 0.1N HCl and neutralizing the solution using 0.1N NaCl. Each of the two resulting reaction mixtures was vortexed for 1 minute and mixed with 40 mL of 100% ethanol. The reaction mixtures were placed in an incubator and rotated for 24 hours at 37° C. (Experiment 44/3), or for 12 days at 37° C. (Experiment 44/4). After the two different incubation periods were completed, the reaction mixtures were centrifuged at 7000 rpm for 15 minutes, the supernatants were removed and the pellets washed with 40 mL 1N HCl followed by washing with 10 mL of DI water. Each washing step included a centrifugation of the sample in order to remove the excess of solvent. Both experiments resulted in a soft gel. In the gel resulting from Experiment 44/3 the DL-glyceraldehyde cross-linked gel had a brighter yellowish color than that of the D(−)-ribose cross-linked gel resulting from Experiment 44/4.

Spectroscopic Characterization of Cross-Linked Polysaccharides

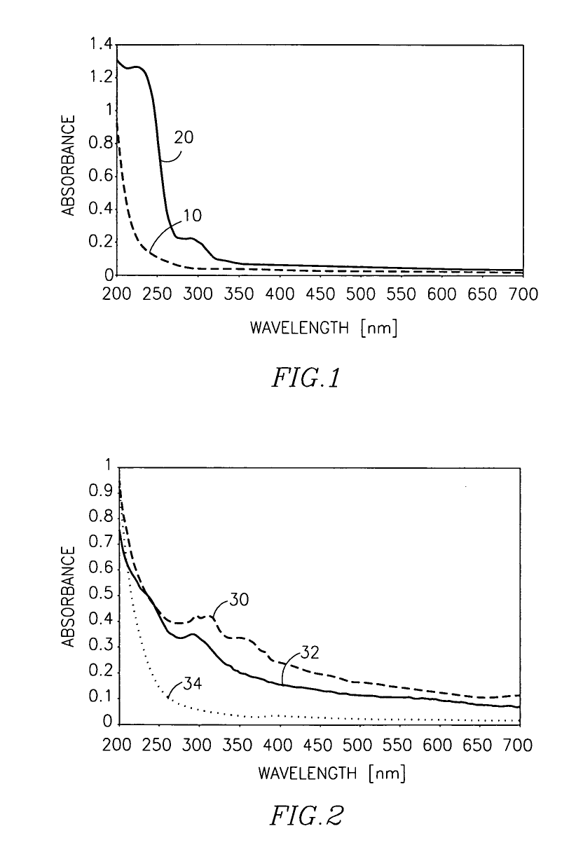

Reference is now made to FIGS. 1-4. In the graphs illustrated in FIGS. 1-3, the vertical axis represents the absorbance of the sample, and the horizontal axis represents the wavelength in nm. In the graph illustrated in FIG. 4, the vertical axis represents the Absorbance of the sample, and the horizontal axis represents the wavenumber (in units of cm⁻¹).

FIG. 1 which is a schematic graph representing the UV-Visible spectra of amino-functionalized hyaluronic acid (AFHA) (represented by the dashed curve 10) and of DL-glyceraldehyde cross-linked AFHA (represented by the solid curve 20), obtained in accordance with an embodiment of the method of the present invention. The dashed line curve represents the spectrum of a sample of amino functionalized HA (AFHA I 150, prepared as disclosed in detail hereinabove) and the solid line curve represents the spectrum of the DL-glyceraldehyde cross-linked product obtained in Experiment 72/1 as described hereinabove. In contrast with the AFHA I 150 sample, the cross-linked polysaccharide sample exhibits strong absorbance in the range of 225-235 nm and 285-355, which may indicate the formation of glycation products in the cross-linking reaction.

FIG. 2 is a schematic graph representing the UV-Visible spectra of D(−)-ribose cross-linked AFHA (represented by the dashed curve 30), D(−)-erythrose cross-linked AFHA (represented by the solid curve 32), and D(−)-arabinose cross-linked AFHA (represented by the dotted curve 34), obtained in accordance with embodiments of the method of the present invention. The sample of the D(−)-ribose cross-linked HA was obtained from the sample of Experiment 67/1. The sample of the D(−)-erythrose cross-linked HA was obtained from the sample of Experiment 67/6. The sample of the D(−)-arabinose cross-linked HA was obtained from the sample of Experiment 67/2.

Peak shifts are possibly a result of the different reducing sugars which were used to cross-link the HA. Each sugar has its own specific chain length and conformation which has an influence on the final advanced glycation endproducts (AGE) formed in the reaction.

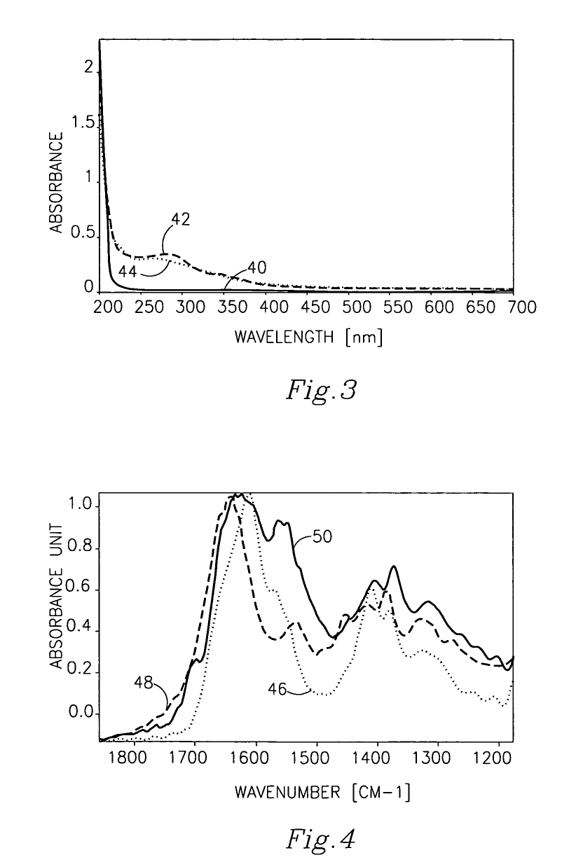

FIG. 3 is a schematic graph representing the UV-Visible spectra of non-cross-linked chitosan (represented by the solid curve 40), D(−)-ribose cross-linked chitosan (represented by the dashed curve 42), and DL-glyceraldehyde cross-linked chitosan (represented by the dotted curve 44), obtained in accordance with embodiments of the method of the present invention. The sample of the non cross-linked chitosan (curve 40) was obtained from Aldrich as indicated in TABLE 1 hereinabove. The sample of the D(−)-ribose cross-linked chitosan (curve 42) was obtained from the sample of Experiment 44/4. The sample of the DL-glyceraldehyde cross-linked chitosan (curve 44) was obtained from the sample of Experiment 44/3.

In contrast to the sample with unmodified (non-cross-linked) chitosan, the two samples of chitosan cross-linked with D(−)-ribose and DL-glyceraldehyde exhibited enhanced absorbance around 290 nm (possibly indicating the typical absorbance of the assumed glycation products and possibly AGE.

FIG. 4 is a schematic graph representing the Fourier Transform Infra-red (FTIR) spectra of hyaluronic acid (represented by the dotted curve 46), AFHA represented by the dashed curve 48 and DL-glyceraldehyde cross-linked AFHA represented by the solid curve 50, in accordance with embodiments of the method of the present invention.

The dotted curve represents the IR spectrum of hyaluronic acid (HA150). The dashed curve illustrates the IR spectrum of the amino functionalized HA (AFHA I 150). The solid curve represents the spectrum of the sample of DL-glyceraldehyde cross-linked HA obtained in Experiment 33/1. The IR spectra of FIG. 4 show the range of absorbance of the carboxyl (COO⁻) groups and overlapping with these absorbance in the range 1610-1550 cm⁻¹ and 1420-1300 cm⁻¹ the absorbance of amide and amine groups (1650 cm⁻¹ and 1560 cm⁻¹, respectively) which may shift to higher or lower wavenumbers, depending on their environment. In addition, an absorbance in the range of 1740-1700 cm⁻¹ appears after the cross-linking. Typically, the absorbance of carboxylic acids as well as the absorbance of amides and amino acids is found in this wavelength range (of 1740-1700 cm⁻¹). Since all of these functional groups exist in the final cross-linked HA, it is difficult to differentiate between them. Generally, significant absorbance spectra changes may be observed in the cross-linking reaction products disclosed herein, which strongly suggests a modification and chemical reaction during the amino-functionalization and the cross-linking procedures used and the formation of glycation products.

Physical Characterization of Cross-Linked Polysaccharides

All characterizations of the cross-linked polysaccharides obtained in the experiments described hereinabove were performed using standard methods on a model HAAKE RheoStress 600 rotational rheometer commercially available from Thermo Electron Corporation GmbH, Germany. In all measurements, a model PP 20 Ti PR rotor was used with a model MPC20/S QF measurement plate cover. The measurements were performed at a Temperature of 23° C.

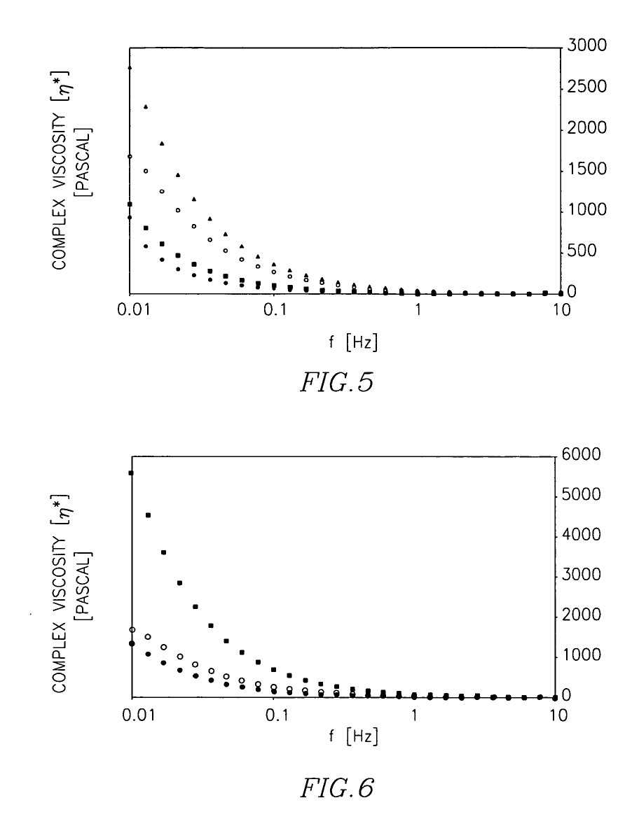

All rheological tests where performed using oscillatory measurements method. 400 μL of the tested material were placed between two chequered plates of the rheometer. Sinusoidal stress was applied to all the samples at a frequency range between 0.01 and 10 Hz. The resulting values of the complex viscosity |η*| are given in Table 2 below. Selected rheology measurement results are also illustrated in more detail in FIGS. 5-7.

TABLE 2

| Experiment No. | Complex viscosity \|η*\| in Pascal |
|---|---|
| or product name | at an oscillating frequency of 0.01 Hz |
| 53/1 | 248 (not extruded) |
| | 387 (extruded through a 20G needle) |
| 53/2 | 526 (not extruded) |
| | 643 (extruded through a 20G needle) |
| 53/3 | 929 (not extruded) |
| | 483 (extruded through a 20G needle) |
| 54/1 | 1094 |
| | 653 (extruded through a 20G needle) |
| 60/1 | 2197 |
| 61/1 | 1554 |
| 62/1 | 2765 |
| 65/3 | 2689 |
| 65/4 | 1663 |
| 65/5 | 623 |
| 67/1 | 1333 (not extruded) |
| | 2391 (extruded 4 times through 27G needle) |
| 67/2 | 5599 (not extruded) |
| | 7036 (extruded 5 times through 27G needle) |
| | 6727 (when extruded 2 times through 30G |
| | needle) |
| 72/1 | 2279 |
| | 3873 (20 hours at 37° C.) |
| 75/1 | 8926 |
| 75/2 | 6606 |
| 75/3 | 6721 |
| 75/4 | 4415 |
| 77/1 | 2279; |
| | 5226 (24 hours at 37° C.) |
| | 5436 (48 hours at 37° C.) |
| | 6863 (20 days at 37° C.) |
| Restylane ® lot: 7349 | 2216 |
| Perlane ® lot: 7064 | 2419 |
| Hylaform ® lot: R0409 | 614 |

It is noted that when multiple measured values of |η*| are presented in the right hand column of TABLE 2, the first indicated value represents the result of measurement of the final reaction product sample as directly taken from the final pellet (or as directly taken from the commercial product syringe for the Restylane®, Perlane® and Hylaform® samples). Other measured |η*| values indicated (within the same row) for the same sample of the same experiment show results obtained from the same sample which was further processed by either extruding the sample through a needle (as indicated in detail within parenthesis after the numerical value) or by further incubating the sample for a specified time period at 37° C. (the precise time period of incubation is specified in the parenthesis following the numerical value).

Reference is now made to FIGS. 5-7 which are schematic graphs illustrating the results of measurements of the rheological properties of different compositions of AFHA based polysaccharide cross-linked with various DL-glyceraldehyde concentrations for various times as compared to the rheological properties of some commercially available hyaluronic acid based matrices. In FIGS. 5-7 the vertical axis represents the complex viscosity (|η*|) in Pascal, and the horizontal axis represents the oscillation frequency in Hz.

In FIG. 5, the filled circles represent experimental data points obtained for the cross-linked matrix of experiment 53/3 (which was cross-linked with 100 mg glyceraldehyde for 24 hours at 37° C.). The filled squares represent experimental data points obtained for the cross-linked matrix of experiment 54/1 (which was cross-linked with 150 mg of DL-glyceraldehyde for 24 hours at 37° C.). The filled triangles represent experimental data points obtained for the cross-linked matrix of experiment 62/1 (which was cross-linked with 300 mg of DL-glyceraldehyde for 24 hours at 37° C.). The open circles represent experimental data points obtained for the commercially obtained Perlane® (lot: 7064) (see TABLE 2 for the exact numerical values).

As may be seen in the graph of FIG. 5, increasing the DL-glyceraldehyde concentration under similar reaction conditions significantly increases the viscosity of the resulting cross-linked polysaccharide. Furthermore, the cross-linked polysaccharide resulting from Experiment 62/1 (in which 300 mg of DL-glyceraldehyde were used in the cross-linking mixture) has complex viscosity values significantly higher than the those of the commercially available hyaluronic acid based Restylane® Perlane® for frequency values below 0.1 Hz.

In FIG. 6, the filled circles represent experimental data points obtained for the cross-linked matrix of experiment 67/1 (which was cross-linked with 50 mg of DL-glyceraldehyde for 48 hours at 37° C.). The filled squares represent experimental data points obtained for the cross-linked matrix of experiment 67/2 (which was cross-linked with 50 mg of DL-glyceraldehyde for 72 hours at 37° C.). The open circles represent experimental data points obtained for the commercially obtained Perlane® (lot: 7064) (see TABLE 2 for the exact numerical values).

As may be seen from FIG. 6, when the incubation time of the cross-linking reaction of AFHA is increased from 48 hours to 72 hours, the complex viscosity values of the resulting HA based cross-linked polysaccharide increases significantly with increasing the cross-linking reaction duration. Moreover, the cross-linked polysaccharide resulting from Experiment 67/2 (in which a 72 hour cross-linking reaction was performed with 50 mg of DL-glyceraldehyde used in the cross-linking mixture) has complex viscosity values significantly higher than the those of the commercially available hyaluronic acid based Restylane®-Perlane® for frequency values below 0.1 Hz.

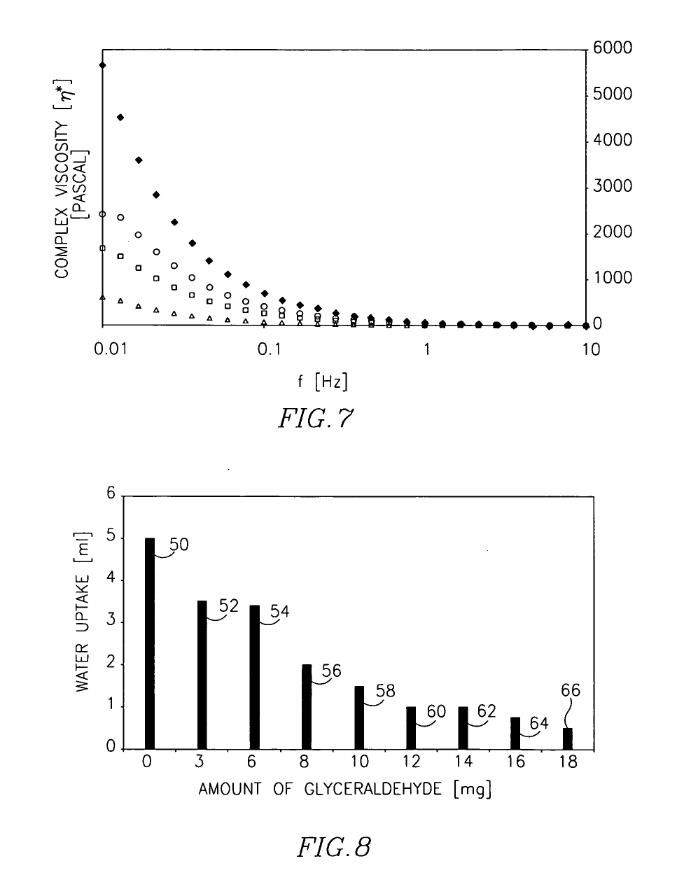

In FIG. 7, the filled rhombus symbols represent experimental data points obtained for the cross-linked matrix of experiment 77/1 (which was cross-linked with 40 mg of DL-glyceraldehyde for three days at 37° C.). The open circles represent experimental data points obtained for commercially obtained Perlane® (lot: 7064). The open squares represent experimental data points obtained for commercially obtained Restylane® (lot: 7349). The open triangles represent experimental data points obtained for the commercially obtained Hylaform® Plus Lot Number R0409 (see TABLE 2 for the exact numerical values).

As may be seen from FIG. 7, when the complex viscosity values of three commercially available hyaluronic acid based injectable gels and the DL-glyceraldehyde cross-linked HA based polysaccharide of Experiment 77/1 are compared, the complex viscosity values measured are consistently and significantly higher for the cross-linked material obtained in Experiment 77/1 across the oscillation frequency range of 0.1-0.01 Hz. For example, at 0.01 Hz, the complex viscosity of the cross-linked material obtained in Experiment 77/1 is more than twice the complex viscosity measured for Restylane® Perlane® Lot No. 7064, more than three times the complex viscosity measured for Restylane®-Lot No. 7349, and more than eight times the complex viscosity measured for Hylaform®-Plus Lot No. R0409.

It will be appreciated by those skilled in the art that such increasing of viscosity values may be advantageously correlated with an improvement of lifting and building capacity of the filler which may be particularly desirable in materials used for esthetic surgery and cosmetic purposes. While the improved gels disclosed herein have such superior viscosity values, they are nonetheless still easily injectable through needles as small as a 30G needle.

Enzymatic Degradation Resistance Assays

Degradation resistance assays were performed using Hyaluronidase digestion and Uronic acid/Carbazole assay method as described in: Carbohydrate Analysis: A Practical Approach, 2nd ed.: M. F. Chaplin and J. F. Kennedy, IRL Press at Oxford University Press, UK, 1994, (ISBN 0-19-963449-1P) pp. 324, which is incorporated herein by reference in its entirety for all purposes.

The results of the hyaluronidase digestion experiments of some the above disclosed experiments are given in FIG. 10 below. Two experiments were performed:

### First Digestion Experiment

1a) Digestion of Cross-Linked HA

Five samples of 200 μL of cross-linked amino-functionalized HA resulting from Experiment 75/3, were each mixed with 250 μL of NaCl (0.9%) solution and 60.8 units of hyaluronidase dissolved in 50 μL of DI water. All samples were incubated at 37° C. Samples were taken at consecutive one hour intervals after starting the digestion, homogenized by vortexing the material for 1 minute and centrifuged at 13000 rpm for 5 min Heraeus “biofuge pico” centrifuge Cat-No. 75003280, using a Heraeus # 3325B rotor (the centrifuge and rotor are commercially available from Kendro Laboratory Products, Germany). 250 μL of the resulting supernatant were used to perform the carbazole assay.

1b) Digestion of Perlane® Lot No. 7064

Five samples of 200 μL of Perlane® (Lot No. 7064) were each mixed with 250 μL NaCl (0.9%) solution and 60.8 units of hyaluronidase dissolved in 50 μL of DI water and the samples were incubated at 37° C. Samples were taken at consecutive I hour intervals after starting the digestion. The removed samples were homogenized by vortexing the material for 1 minute and centrifuged at 13000 rpm for 5 min in the same Heraeus “biofuge pico” centrifuge. 250 μL of the resulting supernatants were used to perform the carbazole assay.

According to the carbazole assay procedure, the absorbance was measured at 525 nm for each sample. Due to the too intensive color reaction in the case of Perlane®, the sample needed to be diluted 1:10 using borate sulfuric acid. The samples at two hours of incubation were rejected in both cases (75/3 experimental product and Perlane®) due to an excessively high temperature during the carbazole procedure) and are therefore not presented in the graph of FIG. 10.

Reference is now made to FIG. 10 which is a schematic graph illustrating the carbazole assay results of digestion by hyaluronidase of DL-glyceraldehyde cross-linked amino-functionalized HA and commercially obtained Perlane®.

The vertical axis of the graph of FIG. 10 represents the absorbance of the tested samples at a wavelength of 525 nm, and the horizontal axis represents the time in hours from the starting of the digestion test. After taking into account the fact that the Perlane® samples (the data points of which are represented by filled squares in FIG. 10) had to be diluted tenfold prior to reading the absorbance (due to an unreadably high absorbance of the non-diluted samples), while the absorbance of the other samples of the amino-functionalized HA resulting from Experiment 75/3 (the data points of which are represented by filled circles in FIG. 10) were read as they were (without diluting), it is apparent that the samples of the cross-linked amino-functionalized HA obtained from Experiment 75/3 had a much higher (at least several fold higher) resistance to degradation by hyaluronidase than the resistance exhibited by Perlane®

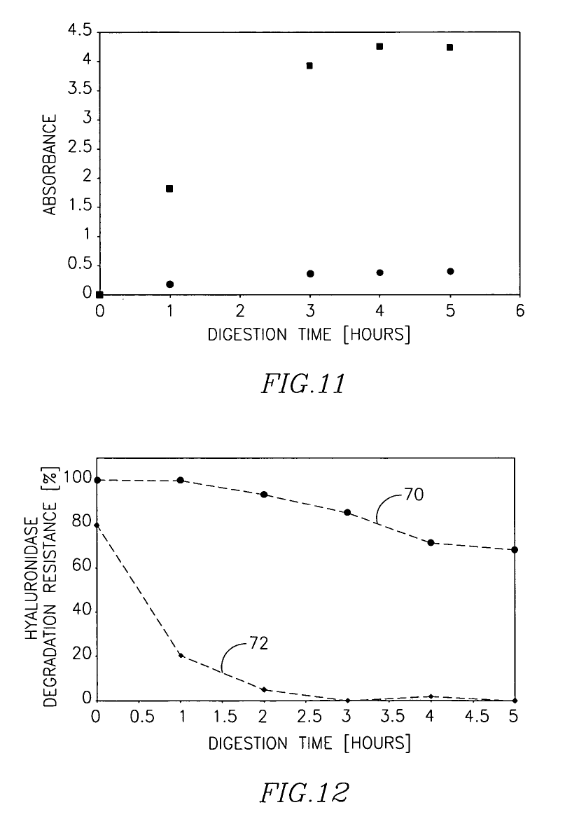

Reference is now made to FIG. 11 which is a schematic graph illustrating the carbazole assay results of FIG. 10 in which the absorbance values for Perlane® (schematically represented by filled squares in FIG. 11) were multiplied by ten to compensate for the 10 fold dilution of the Perlane® test samples. The absorbance values of the samples of the amino-functionalized HA resulting from Experiment 75/3 are schematically represented by filled circles in FIG. 11. The absorbance values of the Perlane® samples resulting from Experiment 75/3 are represented by filled circles in FIG. 11.

While it is well known that it is usually not possible to obtain an accurate value of the absorbance by simply multiplying the absorbance value obtained from a diluted sample, this was simply done to give an approximate impression of the difference between the absorbance values of the cross-linked amino-functionalized HA obtained from Experiment 75/3 and those for Perlane®. Thus, the values represented in FIG. 11 are just a very rough approximation for illustrative purposes only and may not be an accurate representation of the true difference in absorbance between the two materials.

### Second Digestion Experiment

0.1439 mg cross-linked HA or 0.1507 mg Perlane® (Lot No. 7064) were each separately mixed with 250 μL NaCl (0.9%) solution and 60.8 units of hyaluronidase dissolved in 50 μL DI water. The two resulting digestion reaction mixtures were incubated at 37° C. After 4 hours of incubation the two samples were homogenized by vortexing the material for 1 minute and centrifuged at 13000 rpm for 5 min in a Heraeus Biofuge pico centrifuge. The supernatants were removed from both samples and the remaining (non-digested) sample weight was determined for each of the digestion samples. 99.5% of the cross-linked amino-functionalized HA resulting from Experiment 75/3, and 9.3% of Perlane® (Lot No. 7064) remained as sediment after centrifugation and removal of the supernatant. These results strongly corroborate the much higher (in vitro) resistance to hyaluronidase digestion of the amino-functionalized HA resulting from Experiment 75/3 as compared to commercial sample of Perlane®.

The high resistance to hyaluronidase digestion in-vitro of the sugar cross-linked polysaccharide material prepared according to the cross-linking methods disclosed herein indicates that the material may exhibit similarly high resistance to biodegradation in-vivo, which is highly advantageous in matrices used as fillers or bulking agents for tissue augmentation in general and in esthetic treatments in particular as it may increase implant lifetime and may decrease the frequency of needed esthetic treatment, thus reducing the overall cost of treatment and the number and/or frequency of treatments or injections required, resulting in improved patient comfort.

Sample Swelling Tests

Due to the presence of an excess amount of ethanol during the cross-linking process, the sugar cross-linked amino functionalized HA appears in its dehydrated form. In comparison to its hydrated form, the volume of the dehydrated form of the cross-linked HA is negligible. After the described washing procedures (in which the reaction products where re-hydrated by the final washing (for example, in 5 mL of DI water in experiment 35) the resultant gel was transferred into standard test tubes and the volume of the gel (in mL) was determined.

Reference is now made to FIGS. 8-9. FIG. 8 is a schematic bar graph illustrating the results of measuring the swelling behavior of amino functionalized HA cross-linked with various different concentrations of DL-glyceraldehyde, in accordance with an embodiment of the present invention.

The schematic bar diagram of FIG. 8 shows the results of water uptake (swelling) of the DL-glyceraldehyde cross-linked amino-functionalized hyaluronic acid samples obtained in Experiments 35/2, 35/4, 37/4, 37/5, 37/6, 38/1, 38/2 and 38/3 (represented by bars 52, 54, 56, 58, 60, 62, 64, and 66 of FIG. 8, respectively). The leftmost bar 50 of the graph of FIG. 8 represents the result of hydrating AFHA80 in an amount similar to the amount used in the cross-linking experiments but without using DL-glyceraldehyde (this result represents a non-cross-linked AFHA80 sample). For each bar of the graph of FIG. 8, the amount (in mg) of the DL-glyceraldehyde used in the cross-linking of each of the samples is indicated on the horizontal axis of the graph. The volume (in mL) of the tested sample after hydration (by washing) and centrifugation is represented on the vertical axis as a representation of the amount of water-induced swelling of the sample after removal of the ethanol from the reaction mixture by washing. The results of FIG. 8 show a consistent higher sample swelling which is maximal in the non-cross-linked AFHA80 sample and fairly consistently decreases as the amount of cross-linker (DL-glyceraldehyde) in the reaction mixture increases.

FIG. 9 is a schematic graph illustrating the results of measuring the swelling behavior of chitosan cross-linked with various different concentrations of DL-glyceraldehyde, in accordance with an embodiment of the present invention. The schematic bar diagram of FIG. 9 shows the results of water uptake (swelling) of the DL-glyceraldehyde cross-linked chitosan samples obtained in Experiments 40/1, 40/2, and 40/3 (represented by bars 62, 64 and 66, of FIG. 9, respectively). The leftmost bar 60 of the bar graph of FIG. 9 represents the result of hydrating non-cross-linked chitosan in an amount similar to the amount used in the cross-linking EXPERIMENT SERIES 40/1-3 but without using DL-glyceraldehyde (a non-cross-linked chitosan sample). The amount (in mg) of the DL-glyceraldehyde used in the cross-linking of the chitosan samples is indicated on the horizontal axis of the graph. The volume (in mL) of the sample after hydration (by washing) and centrifugation is represented on the vertical axis as a representation of the amount of water-induced swelling of the sample after removal of the ethanol from the reaction mixture by washing. The results of FIG. 9 show a consistent higher sample swelling being maximal in the non-cross-linked chitosan sample which fairly consistently decreases as the amount of cross-linker (DL-glyceraldehyde) in the reaction mixture increases (from 300 mg through 400 mg to 500 mg, in the examples illustrated in FIG. 9).

Composite Matrices Made by Cross-Linking Chitosan and Collagen

Fibrillar collagen was prepared as described in detail in the U.S. Pat. No. 6,682,760, incorporated herein by reference in its entirety for all purposes. The fibrillated collagen was concentrated by centrifugation (4500 rpm).

### Experiment 1

Three different test tubes were prepared each containing 140 mg fibrillated collagen added to a mixture of 14.5 mL of 10 mM PBS buffer (pH 7.36), 5 mL of fibrillation buffer (prepared as disclosed in detail hereinabove), 35 mL 100% Ethanol and 100 mg of D(−)-ribose dissolved in 500 μL of PBS buffer. The reaction mixtures were vortexed.

Three different chitosan solutions a, b and c were prepared as follows:

a) 13.5 mg of chitosan were dissolved in 2.5 mL of 0.1N HCL

b) 27 mg of chitosan were dissolved in 5.0 mL of 0.1N HCL

c) 54 mg of chitosan were dissolved in 10.0 mL of 0.1N HCL

Each of the solutions a, b and c was drop-wise slowly added to one of the collagen/D(−)-ribose mixtures in the test tubes with constant stirring. The reaction mixtures were vortexed for 1 minute, and were rotated in an incubator at 37° C. for 12 days. At the end of the incubation period, the mixtures were centrifuged for 15 minutes at 5000 rpm. All the resulting reaction products had a paste-like consistency. For increasing concentrations of chitosan, the yellowish color of the products also gradually changed from off white to deep yellow. In case c) conglomerates of cross-linked chitosan were found inside the paste.

### Experiment 2

Three different test tubes were prepared each containing 140 mg fibrillated collagen added to a mixture of 17.5 mL of 10 mM PBS buffer, 5 mL of fibrillation buffer, 17.5 mL of 100% ethanol and 33 mg of DL-glyceraldehyde dissolved in 300 μL of 10 mM PBS buffer. The reaction mixtures were vortexed.

Three different chitosan solutions a, b and c were prepared as follows:

a) 13.5 mg of chitosan were dissolved in 2.5 mL of 0.1N HCL.

b) 27 mg of chitosan were dissolved in 5.0 mL of 0.1N HCL.

c) 54 mg of chitosan were dissolved in 10.0 mL of 0.1N HCL.

Each of the chitosan solutions a, b and c above was drop-wise slowly added to one of the collagen/DL-glyceraldehyde reaction mixtures in the test tubes with constant stirring. After an additional vortexing for 1 minute, the reaction mixtures were rotated in an incubator at 37° C. for 24 hours. At the end of the incubation period, the mixtures were centrifuged for 15 minutes at 5000 rpm. All the resulting reaction products had a paste-like consistency. As the concentration of chitosan increased, the yellowish color of the products gradually increased from off-white to bright yellow. In case c) conglomerates of cross-linked chitosan were found inside the paste.

### Experiment 7/1

Five different test tubes were prepared each containing 108 mg fibrillated collagen added to a mixture of 9.8 mL of 100% ethanol and 14 mg of DL-glyceraldehyde dissolved in 700 μL fibrillation buffer and vortexed. The five collagen/DL-glyceraldehyde mixtures were rotated for 6 hours in an incubator at 37° C.

The following five chitosan solutions were also prepared:

a) 53 mg of chitosan were dissolved in 3.2 mL of 0.1N HCL

b) 75.6 mg of chitosan were dissolved in 4.5 mL of 0.1N HCL

c) 107.5 mg of chitosan were dissolved in 6.4 mL of 0.1N HCL

d) 141 mg of chitosan were dissolved in 8.4 mL of 0.1N HCL

e) 161.3 mg of chitosan were dissolved in 9.6 mL of 0.1N HCL

Each of the solutions a, b, c, d, and e was slowly added drop wise to one of the five test tubes containing the collagen/DL-glyceraldehyde mixtures described hereinabove. After an additional vortexing for 1 minute, the mixtures were again placed in an incubator and rotated for 24 hours at 37° C. After the second incubation period ended, the mixtures were centrifuged for 15 min at 5000 rpm.

All the resulting cross-linked products had a paste-like consistency. For increasing concentrations of chitosan, the yellowish color of the products gradually increase from off-white to bright yellow. Sample c) was incubated at 50° C. for 6 hours in an excessive amount of 6N NaOH in order to dissolve the non-cross linked collagen. After three washing (in DI water) and centrifugation steps, the sample was subjected to HCl hydrolysis and analyzed by an amino acid analyzer (in a commercial laboratory) in order to detect hydroxyproline (representing collagen) covalently bounded to chitosan. Hydroxyproline was detected in the samples.

### Experiment 7/2

Three different test tubes were prepared each containing 108 mg of fibrillated collagen added to a mixture of 9.8 mL of 100% ethanol and 14 mg of DL-glyceraldehyde dissolved in 700 μL of fibrillation buffer and vortexed.

The following three chitosan solutions a, b and c were also prepared:

a) 53 mg of chitosan were dissolved in 3.2 mL of 0.1N HCL.

b) 107.5 mg of chitosan were dissolved in 6.4 mL of 0.1N HCL.

c) 161.3 mg of chitosan were dissolved in 9.6 mL of 0.1N HCL.

Each of the solutions a, b and c, was slowly added drop wise to one of the three test tubes containing the collagen/DL-glyceraldehyde mixtures, with constant stirring. After an additional vortexing for 1 minute, the mixtures were rotated for 24 hours in an incubator at 37° C. After the end of the incubation period, the mixtures were centrifuged for 15 min at 5000 rpm. All the resulting reaction products had a paste-like consistency. For increasing concentrations of chitosan, the yellowish color of the products gradually increased from off-white to bright yellow.

Heparin Amino Functionalization

500 mg of Heparin sodium EP (Batch No. 9818030) were dissolved in 300 mL of DI water. 3.0 g of adipic acid dihydrazide (ADH) were added to the mixture. The pH of the resulting solution was adjusted to 4.75 and the solution was stirred until a homogeneous solution was obtained. 400 mg of 1-ethyl-3-(dimethyl aminopropyl)carbodiimide hydrochloride was dissolved in 2.0 mL DI water and added to the mixture and the pH was again adjusted to 4.75 at room temperature. The reaction was monitored by monitoring the pH which was continuously adjusted to 4.75. The reaction mixture was left overnight with stirring. The solution was then transferred into a dialysis tube and subjected to alternating dialysis against DI water and against a DI water/ethanol mixture (4:1 v/v) until no ADH was detected in the dialyzate. The resulting amino-functionalized Heparin sodium EP (Heparin-M) was stored at 4° C. until used.

HA Amino-Functionalization Procedure II

2.4 g of HA 150 (sodium hyaluronate Pharma Grade 150, commercially available as product No. 2222003 from NovaMatrix FMC Biopolymer, Oslo, Norway) having a molecular weight in the range of 1.4-1.8 MDa were dissolved in 2.0 L of DI water and 7.0 g of adipic acid dihydrazide (ADH) were added to the mixture. The pH of the resulting solution was adjusted to pH 4.75 and the solution was stirred until a homogeneous solution was obtained. 760 mg of 1-ethyl-3-(dimethyl aminopropyl)carbodiimide hydrochloride were dissolved in 10.0 mL DI water and added to the mixture and the pH was again adjusted to 4.75 at room temperature. The reaction was monitored by monitoring the change of the pH which was continuously adjusted to pH 4.75. When no change of pH could be detected, the reaction mixture was left for an additional hour or overnight. The solution was then transferred into a dialysis tube and subjected to alternating dialysis against DI water and against a DI water/ethanol mixture (4:1 v/v) until no ADH was detected in the dialyzate. The dialyzed solution was transferred into 3.5 liter of 100% ethanol, 5 g of NaCl were added and the mixture was stirred for one hour. In order to separate the precipitated modified HA, the solution was centrifuged for 20 minutes at 7000 rpm and the supernatant was removed. The resulting amino-functionalized HA (AFHA II) was stored at 4° C. until used.

HA Amino-Functionalization Procedure III

2.5 g of HA 150 (sodium hyaluronate Pharma Grade 150, commercially available as product No. 2222003 from NovaMatrix FMC Biopolymer, Oslo, Norway) having a molecular weight in the range of 1.4-1.8 MDa were dissolved in 2.0 L of DI water and 3.4 g of adipic acid dihydrazide (ADH) were added to the mixture. The pH of the resulting solution was adjusted to 4.75 and the solution was stirred until a homogeneous solution was obtained. 400 mg of 1-ethyl-3-(dimethyl aminopropyl)carbodiimide hydrochloride were dissolved in 10.0 mL DI water and added to the mixture and the pH was again adjusted to 4.75 at room temperature. The reaction was monitored by monitoring the change of the pH which was continuously adjusted to pH 4.75. When no change of pH could be detected, the reaction mixture was left for an additional hour or overnight. The solution was then transferred into a dialysis tube and subjected to alternating dialysis against DI water and against a DI water/ethanol mixture (4:1 v/v) until no ADH was detected in the dialyzate. The dialyzed solution was transferred into 3.5 liter of 100% ethanol, 5 g of NaCl were added and the mixture was stirred for one hour. In order to separate the precipitated modified HA, the solution was centrifuged for 20 minutes at 7000 rpm and the supernatant was removed. The resulting amino-functionalized HA (AFHA III) was stored at 4° C. until used.

HA Amino-Functionalization Procedure IV

2.4 g of HA 150 (sodium hyaluronate Pharma Grade 150, commercially available as product No. 2222003 from NovaMatrix FMC Biopolymer, Oslo, Norway) having a molecular weight in the range of 1.4-1.8 MDa were dissolved in 350 mL of DI water, 5.0 g of adipic acid dihydrazide (ADH) were added to the mixture. The pH of the resulting solution was adjusted to 4.75 and the solution was stirred until a homogeneous solution. 500 mg of 1-ethyl-3-(dimethyl aminopropyl)carbodiimide hydrochloride was dissolved in 10.0 mL DI water and added to the mixture and the pH was again adjusted to 4.75 at room temperature. The reaction was monitored by the change of the pH and continuously adjusted to 4.75. After no change of pH could be detected, the reaction mixture was left for an additional hour or over night. Subsequently, the solution was transferred into a dialysis tube and subjected to alternating dialysis against DI water and against a DI water/ethanol mixture (4:1 v/v) until no ADH was detected in the dialyzate. The dialyzed solution was transferred into 3.5 liter of 100% ethanol, 5 g of NaCl were added and the mixture was stirred for 1 hour. In order to separate the precipitated modified HA, the solution was centrifuged for 20 minutes at 7000 rpm and the supernatant was removed. The resulting amino-functionalized HA (AFHA IV) was stored at 4° C. until used.

### Experiment Series 03/105/1-6

Two separate identical slurries each containing 152 mg AFHA I 150 in 6 mL of 100% ethanol were prepared.

A solution containing 900 mg of D(−)-sorbose dissolved in 9 mL of DI water was added to the first AFHA I 150 slurry by placing the cross-linker (D(−)-sorbose) solution under the AFHA I 150 slurry and vortexing to obtain a homogeneous mixture. The resulting reaction mixture was divided into three equal portions 1, 2 and 3 and each of the resulting portions was placed in an incubator and rotated at 37° C. as follows:

In Experiment 03/105/1, portion 1 was incubated for six (6) days.

In Experiment 03/105/2, portion 2 was incubated for twelve (12) days.

In Experiment 03/105/3, portion 3 was incubated for eighteen (18) days.

A solution containing 900 mg of D(−)-fructose dissolved in 9 mL of DI water was added to the second AFHA I 150 slurry by placing the cross-linker (D(−)-fructose) solution under the AFHA I 150 slurry and vortexing to obtain a homogeneous mixture. The resulting reaction mixture was divided into three equal portions 4, 5 and 6 and each of the resulting portions was placed in an incubator and rotated at 37° C. as follows:

In Experiment 03/105/4, portion 4 was incubated for six (6) days.

In Experiment 03/105/5, portion 5 was incubated for twelve (12) days.

In Experiment 03/105/6, portion 6 was incubated for eighteen (18) days.

After the incubation of the reaction mixtures 1-6 above was completed, 40 mL of DI water were added to each of the reaction mixtures 1-6 and the mixtures were centrifuged at 9000 rpm for 20 minutes. The supernatants were removed and 40 mL of physiological NaCl solution (0.9%) together with 2 mL of PBS buffer solution (10 mM, pH 7.36) were added to each of the resulting pellets and mixed. The mixtures were centrifuged again at 9000 rpm for 20 minutes. The complex viscosity values determined for the resulting pellets of experiments 03/105/1, 03/105/1, 03/105/2, 03/105/3, 03/105/4, 03/105/5 and 03/105/6, and a brief description of some observed characteristics of the pellets are summarized in TABLE 5 hereinafter.

### Experiment Series 03/114/1-4

### Experiment 03/114/1

A slurry containing 50 mg AFHA I 150 in 2 mL of 100% ethanol was prepared. A solution of 50 mg D(−)-fructose in 1.5 mL DI water was added to the slurry by placing the cross-linker solution under the AFHA I 150 slurry and vortexing to obtain a homogeneous mixture. The resulting mixture was poured into 40 mL of 100% ethanol. The reaction mixture was placed into an incubator and rotated for two (2) days at 37° C. After completion of the incubation, 40 mL of DI water were added to the reaction mixture and the mixture was centrifuged at 9000 rpm for 10 minutes.

### Experiment 03/114/2

The experiment was performed as described for experiment 03/114/1 above, except that the reaction mixture was incubated with rotation for four (4) days.

### Experiment 03/114/3

The experiment was performed as described for experiment 03/114/1 above, except that 50 mg of D(−)-sorbose were used instead of D(−)-fructose.

### Experiment 03/114/4

The experiment was performed as described for experiment 03/114/3 above, except that the reaction mixture was incubated with rotation for four (4) days instead of two days.

The complex viscosity values determined for the resulting pellets of experiments 03/114/1, 03/114/2, 03/114/3 and 03/114/4, and a brief description of some observed characteristics of the pellets are summarized in TABLE 5 hereinafter.

### Experiment Series 03/140/1-4

### Experiment 03/140/1

A slurry containing 50 mg AFHA I 150 in 1 mL of 100% ethanol was prepared. A cross-linker solution was prepared by dissolving 300 mg D(−)-ribose in 2.0 mL DI water. The cross-linker solution was placed under the AFFA I 150 slurry and the test tube was vortexed to obtain a homogenous mixture. The mixture was then poured into 40 mL of 100% ethanol. The resulting reaction mixture was placed in an incubator and rotated for 5 days at 37° C. At the end of the incubation period, 40 mL of DI water were added to the reaction mixture and the resulting mixture was centrifuged at 7000 rpm for 20 minutes. The resulting pellet was washed with 40 mL of physiological NaCl solution (0.9%) mixed with 2 mL of PBS buffer solution (10 mM, pH 7.36) and centrifuged at 7000 rpm for 20 minutes. The pellet was then homogenized by extrusion once through an 18G needle and then once through a 21G needle and kept in 40 mL physiological NaCl solution (0.9%) for 6 hours and then centrifuged at 7000 rpm for 30 minutes. The pellet was filtered using a Whatman® filter paper No. 4 (commercially available as Cat. Number 1004 320 from Whatman, USA) and incubated at 37° C. for three days.

### Experiment 03/140/2

The experiment was performed as described for experiment 03/140/1 above, except that the cross-linker solution included 50 mg of D(+)-sorbose dissolved in 2.0 mL DI water.

### Experiment 03/140/3

The experiment was performed as described for experiment 03/140/1 described above, except that the cross-linker solution included 50 mg of L(+)-fructose dissolved in 2.0 mL DI water.

### Experiment 03/140/4

The experiment was performed as described for experiment 03/140/1 above, except that the cross-linker solution included 300 mg D(+) glucose dissolved in 2.0 mL DI water, and the pellet was not filtered using filter paper.

The complex viscosity values determined for the resulting pellets of experiments 03/140/1, 03/140/2, 03/140/3 and 03/140/4, and a brief description of some observed characteristics of the pellets are summarized in TABLE 5 hereinafter.

### Experiment 03/140/6

A slurry containing 50 mg AFHA I 150 in 1 mL of 100% ethanol was prepared. A cross-linker solution was prepared by dissolving 300 mg D-ribose-5-phosphate disodium salt dehydrate in 2.0 mL of DI water. The AFHA I 150 slurry was mixed with the cross-linker solution by placing the cross-linker solution under the AFHA I 150 slurry layer and vortexing to obtain a homogeneous mixture. The resulting mixture was poured into 40 mL of 100% ethanol. The reaction mixture was then placed in an incubator and rotated for 5 days at 37° C. At the end of the incubation period, 40 mL of DI water were added to the reaction mixture, and the resulting mixture was shaken and centrifuged at 7000 rpm for 5 minutes. The supernatant was removed and the pellet was washed twice with 40 mL of physiological NaCl solution (0.9%) mixed with 2 mL of PBS buffer solution (10 mM, pH 7.36) and centrifuged at 7000 rpm for 5 minutes. The complex viscosity value determined for the resulting pellets and a brief description of some observed characteristics of the pellets are summarized in TABLE 5 hereinafter.

The results of experiment 03/140/6 demonstrate that the use of reducing sugars for cross-linking amino-functionalized polysaccharides is not limited to using simple reducing sugars and that various different reducing sugar derivatives may also be successfully used for obtaining the cross-linked polysaccharide matrices and composite matrices of the present invention.

### Experiment Series 03/110/1-4

### Experiment 03/110/1

A slurry containing 50 mg AFHA I 150 in 5 mL of 100% ethanol was prepared. The slurry was mixed with a cross-linker solution containing 160 mg of DL-glyceraldehyde dissolved in 8 mL DI water by placing the cross-linker solution under the slurry and vortexing to obtain a homogenous mixture. 6.5 mL of the resulting mixture were poured into 40 mL of 100% ethanol and vortexing. The resulting reaction mixture were placed in an incubator and rotated for one day at 37° C. At the end of the incubation period, 40 mL of DI water and 2 mL PBS buffer solution (10 mM, pH 7.36) were added to the mixture with mixing and the resulting mixture were centrifuged at 9000 rpm for 20 minutes to obtain a pellet.

### Experiment 03/110/2

The experiment was performed as described for experiment 03/110/1 above, except that 40 mL of 1-hexanol were used instead of 40 mL of 100% ethanol.

### Experiment 03/110/3

The experiment was performed as described for experiment 03/110/1 above, except that 40 mL of 1-butanol were used instead of 40 mL of 100% ethanol.

### Experiment 03/110/4

The experiment was performed as described for experiment 03/110/1 above, except that 40 mL of 2-propanol were used instead of 40 mL of 100% ethanol.

The complex viscosity values determined for the resulting pellets of experiments 03/110/4, 03/110/4, 03/110/4 and 03/110/4 and a brief description of some observed characteristics of the pellets are summarized in TABLE 5 hereinafter.

### Experiment Series 03/131/2-5

### Experiment 03/131/2

A slurry containing 50 mg AFHA I 150 in 2 mL of 100% ethanol was prepared. The slurry was mixed with a cross-linker solution containing 140 mg of DL-glyceraldehyde dissolved in 6 mL DI water by placing the cross-linker solution under the slurry and vortexing to obtain a homogenous mixture. 3.5 mL of the resulting mixture were poured into 40 mL of ethyl acetate and the resulting reaction mixture were placed in an incubator and rotated for one day at 37° C. At the end of the incubation period, 40 mL of physiological NaCl solution (0.9%) were added to the mixture with mixing and the resulting mixture were centrifuged at 7000 rpm for 20 minutes and the supernatant removed to obtain a pellet.

### Experiment 03/131/3

The experiment was performed as described for experiment 03/131/2 above, except that 40 mL of acetone (dimethyl ketone) were used instead of 40 mL of ethyl acetate.

### Experiment 03/131/4

The experiment was performed as described for experiment 03/131/2 described above, except that 40 mL of 1-hexanol were used instead of 40 mL of ethyl acetate

### Experiment 03/131/5

A slurry containing 50 mg AFHA I 150 in 2 mL of 100% ethanol was prepared. The slurry was mixed with a cross-linker solution containing 140 mg of DL-glyceraldehyde dissolved in 6 mL DI water by placing the cross-linker solution under the slurry and vortexing to obtain a homogenous mixture. 8.0 mL of the resulting mixture were poured into 40 mL of toluene and the resulting reaction mixture were placed in an incubator and rotated for six (6) days at 37° C. At the end of the incubation period the toluene was washed out by adding 30 mL of 100% ethanol to the reaction mixture, mixing and centrifugation at 7000 rpm for 20 minutes. The ethanol washing and centrifugation was repeated two more times. The resulting pellet was washed three times with a mixture of 25 mL DI water and 20 mL of physiological NaCl solution (0.9%) and centrifugation at 7000 rpm for 20 min. The final washing step was performed by re-suspending the pellet in 40 mL of physiological NaCl solution (0.9%) mixed with 2 mL of PBS buffer solution (10 mM, pH 7.36) and centrifugation at 7000 rpm for 20 minutes. The supernatant was removed and the pellet kept for testing.

The complex viscosity values determined for the resulting pellets of experiments 03/1310/2, 03/131/3, 03/131/4 and 03/131/5 and a brief description of some observed characteristics of the pellets are summarized in TABLE 5 hereinafter.

### Experiment 03/146/2

A slurry of 100 mg AFHA I 150 in 2 mL of 100% ethanol was prepared. 100 mg DL-glyceraldehyde was dissolved in 40.0 mL of PBS buffer solution (10 mM, pH 7.36). The AFHA I 150 slurry was mixed with the cross-linker solution by placing the cross-linker solution under the AFHA I 150 slurry and vortexing to obtain a homogeneous mixture. The resulting mixture was placed into an incubator and rotated for 1 day at 37° C. At the end of the incubation period, the mixture was centrifuged at 10,000 rpm for 15 minutes, the supernatant was removed, the pellet was re-suspended in 40 mL DI water and the resulting suspension was left for 12 hours at room temperature. The mixture was then centrifuged at 7000 rpm for 10 minutes and the pellet was washed twice by re-suspension in 40 mL of physiological NaCl solution (0.9%) mixed with 2 mL of PBS buffer solution (10 mM, pH 7.36) mixing and centrifugation at 7000 rpm for 10 minutes. The resultant pellet was placed into an incubator for 3 days at a temperature of 37° C. The complex viscosity values determined for the resulting pellet and a brief description of some observed characteristics of the pellets are summarized in TABLE 5 hereinafter.

### Experiment Series 05/08/2-4

### Experiment 05/08/2

A slurry containing 50 mg AFHA I 150 in 2 mL of 100% ethanol was prepared. 100 mg DL-glyceraldehyde were dissolved in 3 mL DI water. The AFHA I 150 slurry was mixed with the cross-linker solution by placing the cross-linker solution under the AFHA I 150 slurry and vortexing to obtain a homogeneous mixture. 5.0 mL of the resulting mixture was poured into 40 mL of dichloromethane and the resulting reaction mixture were placed in an incubator and rotated for one (1) day at 37° C. At the end of the incubation period 35 mL of PBS buffer solution (10 mM, pH 7.36) were added to the material and mixed and the resulting suspension was centrifuged at 7000 rpm for 15 minutes and the supernatant removed. The pellet was reserved for testing.

### Experiment 05/08/3

The experiment was performed as described for Experiment 05/098/2 above, except that 40 mL of Hexane were used instead of 40 mL of dichloromethane.

### Experiment 05/08/4

A slurry containing 50 mg AFHA I 150 in 2 mL of 100% ethanol was prepared. 100 mg DL-glyceraldehyde were dissolved in 3 mL DI water. The AFHA I 150 slurry was mixed with the cross-linker solution by placing the cross-linker solution under the AFHA I 150 slurry and vortexing to obtain a homogeneous mixture. 5.0 mL of the resulting mixture was poured into 40 mL of diethyl ether and the resulting reaction mixture was placed into a water bath and shaken for 2 day at 30° C. At the end of the water-bath incubation period, 35 mL of PBS buffer solution (10 mM, pH 7.36) were added to the resulting material and mixed to suspend the material. The resulting suspension was centrifuged at 7000 rpm for 15 minutes and the supernatant removed. The final washing step was done with 40 mL of PBS buffer solution (10 mM, pH 7.36). The mixture was then centrifuged at 20000 rpm for 45 minutes and the supernatant removed.

The complex viscosity values determined for the pellets obtained in experiments 05/08/2, 05/08/3 and 05/08/4 and some observed characteristics of the pellets are summarized in TABLE 5 hereinafter.

Additional Examples of Composite Matrices Including HA

Porcine fibrillar collagen was prepared as described in detail in the U.S. Pat. No. 6,682,760.

### Experiment 03/94/2

A slurry of 80 mg AFHA I 150 in 5 mL of 100% ethanol was prepared. The cross-linker solution included 40 mg DL-glyceraldehyde dissolved in 2.5 mL DI water. The AFHA I 150 slurry was mixed with the cross-linker solution by placing the cross-linker solution under the AFHA I 150 slurry. Additionally, 0.4 mL of fibrillated collagen stock solution (having a concentration of 35 mg/mL of fibrillation buffer) were added to a mixture and the resulting mixture was vortexed to obtain a homogeneous mixture. The resulting mixture was added into 40 mL of 100% ethanol. The resulting reaction mixture was placed into an incubator and rotated for 3 days at 37° C. At the end of the incubation period the mixture was centrifuged at 9000 rpm for 20 minutes and the supernatant was removed. The remaining pellet was washed with 40 mL DI water and centrifuged at 20000 rpm for 30 minutes. The resultant pellet was combined with 25 mL of physiological NaCl solution (0.9% NaCl) and turrax-homogenized at 24000 RPM for 1 minute. The homogenized mixture was centrifuged at 9000 rpm for 20 minutes and the supernatant was removed. The resulting pellet was reserved for testing. The complex viscosity values determined for the resulting pellet and a brief description of some observed characteristics of the pellet of the resulting composite matrix are summarized in TABLE 5 hereinafter.

### Experiment Series 04137123-29

A slurry of AFHA I 150 in 100% ethanol was prepared according to TABLE 3. DL-glyceraldehyde was dissolved in 1.0 mL DI water in an amount as disclosed in TABLE 3 below. The AFHA I 150 slurry was slowly added with continuous vortexing to 2.0 mL of a solution of fibrillated porcine collagen (having a concentration of 35 mg collagen per mL of fibrillation buffer) the total amount of collagen in each experiment is given in TABLE 3. Subsequently, the 1 mL of cross-linker solution was added to the collagen/AHFA mixture and the combined mixture was turrax-homogenized at 24000 RPM for 0.5 minute to obtain a homogeneous mixture. The homogenized mixture was added into 40 mL of 100% ethanol. The resulting reaction mixture was placed into an incubator and rotated overnight at 37° C. At the end of the incubation period the supernatant was removed. The material was washed once with 40 mL DI water and centrifuged at 6000 rpm for 15 minutes and twice with 40 mL PBS buffer solution (10 mM, pH 7.36) with centrifugation at 6000 rpm for 15 minutes. The exact quantities of the materials used in preparing the different reaction mixtures for experiments 04/37/23, 04/37/24, 04/37/25, 04/37/26, 04/37/27, 04/37/28 and 04/37/29 and the experimentally determined values of the complex viscosity are summarized in TABLE 3 below.

### Experiments 04/39/30, 04/41/31, 04/44/32, 04/48/34, 04/52/35 and 04/52/36

A slurry of AFHA I 150 in 100% ethanol was prepared. The composition of the slurry for each experiment is detailed in TABLE 3 hereinafter. DL-glyceraldehyde (used as cross-linker) was dissolved in 1.0 mL DI water in an amount specified in TABLE 3. The AFHA I 150 slurry was mixed with an amount of fibrillated porcine collagen suspended in 2 mL PBS buffer solution (10 mM, pH 7.36) by slow adding with vortexing of the collagen solution to the AHFA solution. The amount of fibrillated collagen used in each experiment is indicated in TABLE 3. The cross-linker (DL-glyceraldehyde) solution was then added to the collagen/AHFA mixture with mixing. The resulting reaction mixtures for all the experiments were all turrax-homogenized at 24000 RPM for 0.5 minute to obtain a homogeneous mixture. 40 mL of 100% ethanol were added to each of the resulting reaction mixtures. The resulting mixtures were placed into an incubator and rotated overnight at 37° C. After the end of the incubation period the supernatants were removed.

Each of the resulting pellets was washed once with 40 mL DI water and centrifuged at 6000 rpm for 15 minutes and then washed twice with 40 mL PBS buffer solution (10 mM, pH 7.36) and centrifuged at 6000 rpm for 15 minutes. Each of the washed pellets was then mixed with 25 mL of physiological NaCl solution and turrax-homogenized at 24000 RPM for 0.5 minute. After the Turrax homogenization, each of the homogenized mixtures was centrifuged at 6000 rpm for 15 minutes and the supernatant was removed. The resulting pellets were tested. The complex viscosity of each one of the resulting pellets was determined as disclosed in detail hereinabove. The quantities of the materials used in preparing the different reaction mixtures and the experimentally determined values of the complex viscosity are summarized in TABLE 3.

TABLE 3

| | Amount | | | | |
|---|---|---|---|---|---|
| | of | Amount | Amount | Amount of | Complex |
| | AFHA I | of | of | DL- | Viscosity at |
| Experiment | 150 | ethanol | collagen | glyceraldehyde | 0.01 Hz |
| Number | [mg] | [mL] | [mg] | [mg] | [Pa * s] |
| 04/37/23 | 6.0 | 0.2 | 50.0 | 50.0 | 1382 |
| 04/37/24 | 10.0 | 0.4 | 50.0 | 50.0 | 2313 |
| 04/37/25 | 18.5 | 0.7 | 50.0 | 50.0 | 1403 |
| 04/37/26 | 33.3 | 1.3 | 50.0 | 50.0 | 3023 |
| 04/37/27 | 56.4 | 2.3 | 50.0 | 50.0 | 2041 |
| 04/27/28 | 86.8 | 3.5 | 50.0 | 50.0 | 2738 |
| 04/37/29 | 112.6 | 4.5 | 50.0 | 50.0 | 2154 |
| 04/44/32 | 160.0 | 4.0 | 40.0 | 240.0 | 3478 |
| 04/41/31 | 100.0 | 3.0 | 11.1 | 100.0 | 4744 |
| 04/48/34 | 185.3 | 1.3 | 9.7 | 278.0 | 4035 |
| 04/52/35 | 100.0 | 2.0 | 0.0 | 150.0 | 6451 |
| 04/52/36 | 100.0 | 2.0 | 42.3 | 150.0 | 5417 |

### Experiment 04/55/1

A slurry of 150 mg AFHA IV 150 in 2 mL 100% ethanol was prepared. 150 mg of D(−)-fructose were dissolved in 5.0 mL of fibrillated porcine collagen (having a concentration of approximately 3 mg collagen per mL of fibrillation buffer solution). The AFHA I 150 slurry was mixed with fibrillated collagen/D(−)-fructose suspension with continuous vortexing. The resulting mixture was turrax-homogenized at 24000 RPM for 0.5 minute to obtain a homogeneous mixture. The homogenized mixture was added into 35 mL of 100% ethanol and 0.5 mL acetic acid (10% v/v). The resulting reaction mixture was placed in an incubator and rotated for 6 hours at 37° C. At the end of the incubation period the supernatant was removed. The remaining pellet was mixed with 25 mL physiological NaCl solution and turrax-homogenized at 24000 RPM for 0.5 minute. The homogenized mixture was centrifuged at 6000 rpm for 15 minutes and the supernatant was removed. The resultant pellet was washed once with 40 mL DI water and centrifuged at 6000 rpm for 15 minutes and then washed twice with 40 mL PBS buffer solution (10 mM, pH 7.36) and centrifuged at 6000 rpm for 15 minutes. The pellet was incubated for 3 days at 37° C.

The complex viscosity value experimentally determined for the resulting pellet and a brief description of some observed characteristics of the pellet are summarized in TABLE 5 hereinafter.

### Experiment 03/145/2

A slurry containing 150 mg AFHA I 150 in 2 mL of 100% ethanol was prepared. 105 mg DL-glyceraldehyde and 5.0 mg cytochrome C from bovine heart were dissolved in 3.0 mL DI water. The AFHA I 150 slurry was mixed with the cross-linker and protein solution by placing the cross-linker solution under the AFHA I 150 slurry and vortexing to obtain a homogeneous mixture. The vortexed mixture was added to 40 mL of ethanol. The resulting mixture was placed into an incubator and rotated for 2 days at 37° C. At the end of the incubation period the supernatant was removed and the resulting material was washed three times by re-suspending in 40 mL of physiological NaCl solution, shaking and centrifugation at 7000 rpm for 10 minutes. The resultant pellet was homogenized by successive extrusion (once per needle) though 18G, 22G and 25G needles. After the extrusion, the material was washed with 40 mL of physiological NaCl solution (0.9%) and centrifuged at 7000 rpm for 10 minutes. The complex viscosity values determined for the resulting pellet and a brief description of some observed characteristics of the resulting pellet are summarized in TABLE 5 hereinafter.

The results of experiment 03/145/2 demonstrate that proteins other than collagen may be successfully cross-linked to amino-functionalized polysaccharides by glycation. It also demonstrates that proteins which are substantially different than collagen may be used in forming the composite cross-linked matrices of the present invention.

The forming of such protein/amino-polysaccharide glycated matrices may be advantageous not only for modifying the rheological properties of the resulting composite matrices by choosing different proteins, but may also be useful for advantageously incorporating biologically active proteins (such as, but not limited to, enzymes, growth promoting or growth inhibiting proteins, various signaling proteins and peptides and the like) into the matrices.

### Experiment 03/146/1

A slurry containing 150 mg AFHA I 150 in 2 mL of 100% ethanol was prepared. 105 mg DL-glyceraldehyde and 3 mL Heparin-M (approximately 40 mg) were dissolved in 3.0 mL DI water. The AFHA I 150 slurry was mixed with the cross-linker solution containing the heparin by placing the cross-linker solution under the AFHA I 150 slurry and vortexing to obtain a homogeneous mixture.

The mixture was added into 40 mL of 100% ethanol. The resulting reaction mixture was placed into an incubator and rotated for 2 days at 37° C. At the end of the incubation period the supernatant was removed and the resulting material was washed twice by mixing with 40 mL of physiological NaCl solution (0.9%) combined with 2 mL of PBS buffer solution (10 mM, pH 7.36), shaking and centrifugation at 7000 rpm for 10 minutes. The resultant pellet was homogenized by successive extrusion through 18G, 22G and 25G needles (once per needle) and placed into an incubator at 37° C. for 3 days. The resulting material was a creamy but firm, opaque gel.

The complex viscosity values determined for the resulting pellets and a brief description of some observed characteristics of the pellets are summarized in TABLE 5 hereinafter.

The results of experiment 03/146/1 demonstrate that the cross-linking with reducing sugars may be applied to various mixtures of different amino-polysaccharides and amino-functionalized polysaccharides (which contain amino groups capable of being cross-linked by reducing sugars) and their derivatives. Such mixed cross-linked matrices may be advantageous because it may be possible to control and modify the physical, chemical and biological properties of such mixed membranes by controlling the specific types and/or the ratio of the different polysaccharides within the cross-linked matrix.

### Experiment Series 05/02/1-2

### Experiment 05/02/1

A slurry containing 150 mg AFHA II 150 in 2 mL of 100% ethanol was prepared. A solution containing 50 mg DL-glyceraldehyde dissolved in 10 mL DI water was mixed with the AFHA II 150 solution by placing the cross-linker solution under the slurry and vortexing to obtain a homogenous mixture. The resulting mixture was poured into 40 mL of 100% ethanol. The resulting reaction mixture was placed into an incubator and rotated for 5 hours at 37° C. At the end of the incubation period the supernatant was removed and the remaining pellet was washed with 35 mL DI water and centrifuged at 7000 rpm for 10 minutes. The resulting pellet was washed twice with 40 mL of physiological NaCl solution mixed with 2 mL of PBS buffer solution (10 mM, pH 7.36), re-suspended and centrifuged at 7000 rpm for 10 minutes. The result was about 30 mL of a soft, transparent gel. This gel was washed four (4) times by re-suspending in 15 mL of 100% ethanol and centrifugation at 10000 rpm for 30 minutes. The resulting pellet was transferred into 35 mL of 100% ethanol mixed with 0.5 mL acetic acid solution (10% in DI water) and the mixture was placed in an incubator at 37° C. and rotated for 24 hours. At the end of the incubation the supernatant was removed. The remaining material was washed with DI water and left at 37° C. for one hour. The sample was then centrifuged at 10000 rpm for 30 minutes. The resulting pellet was washed with 40 mL of physiological NaCl solution mixed with 2 mL of PBS buffer solution (10 mM, pH 7.36), shaken and centrifuged at 10000 rpm for 15 minutes. The pellet was homogenized by sequentially passing through 18G, 20G, 22G, 25G, 27G and 30G needles, washed with 40 mL of physiological NaCl solution mixed with 2 mL of PBS buffer solution (10 mM, pH 7.36), shaken and centrifuged at 10000 rpm for 5 minutes. The pellet was transferred into a syringe and incubated for 3 days at 37° C. After the incubation, the material was tested for determining the complex viscosity.

### Experiment 05/02/2

The experiment was performed as described in experiment 05/02/1 hereinabove, except that the cross-linker solution used included 100 mg D(−)-fructose dissolved in 10 mL DI water. The first incubation step yielded 40 mL of a soft, transparent gel.

The complex viscosity values determined for the final pellets obtained in experiments 05/02/1 and 05/02/2 and a brief description of some observed characteristics of the pellets are summarized in TABLE 5 hereinafter.

### Experiment 05/15/1

A slurry of 150 mg AFHA III 150 in 2 mL of 100% ethanol was prepared. The cross-linker solution included 150 mg D(−)-fructose dissolved in 10 mL DI water. The AFHA III 150 slurry was mixed with the cross-linker solution by placing the cross-linker solution under the AFHA III 150 slurry and vortexing to obtain a homogeneous mixture. The resulting mixture was pored into 40 mL of 100% ethanol. The reaction mixture was then placed into an incubator and rotated for 12 hours at 37° C. and then for an additional two (2) days at room temperature. At the end of the incubation the supernatant was removed. The remaining material was washed with 40 mL DI water mixed with 2 mL of PBS buffer solution (10 mM, pH 7.36) and centrifuged at 8000 rpm for 15 minutes. The resulting pellet was washed with 30 mL of physiological NaCl solution mixed with 2 mL of PBS buffer solution (10 mM, pH 7.36), shaken and centrifuged at 10000 rpm for 15 minutes. The sample was filtered using a Whatman® filter paper No. 113 (Catalogue. Number 1113 320). After filtration, the sample was incubated at 37° C. for 3 days and tested. The experiment yielded about 4.0 mL of slightly opaque gel. The complex viscosity values determined for the final pellets obtained in experiment 05/15/1 and a brief description of some observed characteristics of the pellet is summarized in TABLE 5 hereinafter.

### Experiment 05/18/1

A slurry of 150 mg AFHA III 150 in 2 mL of 100% ethanol was prepared. The cross-linker was 150 mg D(−)-fructose dissolved in 7 mL DI water. The AFHA III 150 slurry was mixed with the cross-linker solution by placing the cross-linker solution under the AFHA III 150 slurry and vortexing to obtain a homogeneous mixture. The mixture was poured into 40 mL of 100% ethanol mixed with 0.5 mL of acetic acid (10% in DI water). The resulting mixture was placed into an incubator and rotated for 12 hours at 37° C. and then for two (2) additional days at room temperature. After the incubation the supernatant was removed. The remaining material was washed with 40 mL DI water mixed with 2 mL of PBS buffer solution (10 mM, pH 7.36) and centrifuged at 8000 rpm for 15 minutes. The resulting pellet was washed with 30 mL of physiological NaCl solution mixed with 2 mL of PBS buffer solution (10 mM, pH 7.36), shaken and centrifuged at 10000 rpm for 15 minutes. The sample was filtered using a Whatman® filter paper No. 113 and homogenized by extruding through a 18 gauge needle. After the homogenization, the sample was incubated at 37° C. for 3 days. The complex viscosity values determined for the resulting pellets and a brief description of some observed characteristics of the pellets are summarized in TABLE 5 hereinafter.

### Experiment Series 05/22/1-4

Four slurries (slurries 1-4), each containing 75 mg AFHA IV 150 in 2 mL of 100% ethanol were prepared. Two different cross-linker solutions were then prepared as follows:

A. 220 mg of D(−)-fructose were dissolved in 7 mL of DI water.

B. 140 mg of D(−)-fructose in 7 mL of DI water.

The AFHA IV 150 slurries of samples 1 and 3 (of experiments 05/22/1 and 05/22/3, respectively) were each mixed with 3.5 mL of cross-linker solution A., and the AFHA IV 150 slurries of samples 2 and 4 (of experiments 05/22/2 and 05/22/4, respectively) were mixed with 3.5 mL cross-linker solution B.

The mixing in all four samples was performed by adding the cross-linker solution to the AFHA IV 150 slurry with continuous vortexing to obtain a homogeneous mixture.

Samples number 1 and 2 (of experiments 05/22/1 and 05/22/2, respectively) were turrax-homogenized at 24000 RPM for 0.5 minute. Each of the four mixtures mixture was separately poured into 40 mL of 100% ethanol. The resulting four reaction mixtures were placed into an incubator and rotated for 2 days at 37° C. After the incubation the supernatants were removed. The remaining pellets were each washed with 40 mL physiological NaCl solution mixed with 2 mL of PBS buffer solution (10 mM, pH 7.36) and centrifuged at 3000 rpm (centrifuge: Kubota KS-8000, swinging bucket rotor RS 3000/6, Stainless steel bucket 53592) for 5 minutes. The resulting four pellets were each washed with 40 mL of physiological NaCl solution mixed with 2 mL of PBS buffer solution (10 mM, pH 7.36), shaken and centrifuged at 3000 rpm for 15 minutes. The resulting four pellets were incubated at 37° C. for 3 days. The complex viscosity values determined for the resulting pellets of experiments 05/22/1, 05/22/2, 05/22/1 and 05/22/2, and a brief description of some observed characteristics of the pellets are summarized in TABLE 5 hereinafter.

### Experiment Series 05/23/1,2

Two slurries of 75 mg AFHA IV 150 in 2 mL of 100% ethanol were prepared. 70 mg D(−)-fructose was dissolved in 5 mL DI water.

Each AFHA IV 150 slurry was unified with 2.5 mL cross-linker solution by adding the cross-linker solution during vortex of the AFHA IV 150 slurries to reach a homogeneous mixture. Sample number 2 was turrax-homogenized at 24000 RPM for 0.5 minute. Each mixture was added into 40 mL of ethanol. The resulting mixtures were transferred into an incubator and rotated for 2 days at 37° C. Afterwards, the supernatant was removed. The remaining material was washed with 40 mL physiological NaCl solution together with 2 mL of PBS buffer solution (10 mM, pH 7.36) and centrifuged at 3000 rpm (centrifuge: Kubota KS-8000, swinging bucket rotor RS 3000/6, Stainless steel bucket 53592) for 5 minutes. The resultant pellet was washed with 40 mL of physiological NaCl solution together with 2 mL of PBS buffer solution (10 mM, pH 7.36), shaken and centrifuged at 3000 rpm for 15 minutes. The samples were then incubated at 37° C. for 3 days. The complex viscosity values determined for the resulting pellets and a brief description of some observed characteristics of the pellets are summarized in TABLE 5 hereinafter.

Enzymatic Degradation Resistance Assays

Degradation resistance assays were performed using hyaluronidase digestion and Uronic acid/Carbazole assay method as described in: Carbohydrate Analysis: A Practical Approach, 2nd ed.: M. F. Chaplin and J. F. Kennedy, IRL Press at Oxford University Press, UK, 1994, (ISBN 0-19-963449-1P) pp. 324, which is incorporated herein by reference in its entirety for all purposes.

The results of the hyaluronidase digestion experiments of some the above disclosed experiments are given in FIG. 10. Two digestion experiments were performed:

1a) Digestion of the Cross-Linked HA

Five samples of approximately 100 μL of cross-linked amino-functionalized HA resulting from Experiment 05/02/02 (having a concentration of 25.6 mg AFHA II 150 cross-linked with D(−)-fructose per mL) were each mixed with 500 μL of PBS buffer solution (10 mM, pH 7.36) and 10 units of hyaluronidase dissolved in 10 μL of DI water. All samples were incubated at 37° C. The exact sample volumes are given in the second column of TABLE 4A below. The samples were taken out from incubation at consecutive one hour intervals after starting the digestion, each removed sample was homogenized by vortexing the material for one minute and centrifuging at 13000 rpm for 15 min in a Heraeus “biofuge pico” centrifuge (Cat-No. 75003280, using a Heraeus # 3325B rotor, the centrifuge and rotor are commercially available from Kendro Laboratory Products, Germany). 25 μL of the resulting supernatant and 225 μL of PBS buffer solution (10 mM, pH 7.36) were used to perform the carbazole assay. The results of the cross-linked HA digestion test are summarized in TABLE 4A.

1b) Digestion of Perlane® Lot No. 7576

Five samples of approximately 100 μL of Perlane® (Lot No. 7576) having a concentration of 20 mg/mL were each mixed with 500 μL of PBS buffer solution (10 mM, pH 7.36) and 10 units of hyaluronidase dissolved in 10 μL of DI water and the samples were incubated at 37° C. The exact volume of the samples are given in the second column of TABLE 4B below. The samples were taken out of incubation at consecutive one hour intervals after starting the digestion. Each of the removed samples was homogenized by vortexing the material for one minute and centrifugation at 13000 rpm for 15 min in the same Heraeus “biofuge pico” centrifuge. 25 μL of the resulting supernatants and 225 μL of PBS buffer solution (10 mM, pH 7.36) were used to perform the carbazole assay. According to the carbazole assay procedure, the absorbance was measured at 525 nm for each sample.

Reference is now made to FIG. 12 which is a schematic graph illustrating the % resistance to hyaluronidase degradation in vitro of an exemplary sample of the D(−)-fructose cross-linked amino-functionalized HA matrix from Experiment May 2, 2002 and of a commercially obtained sample of Perlane® as a function of digestion time (in hours). The vertical axis of the graph of FIG. 12 represents the resistance to hyaluronidase degradation (the amount of HA remaining after the specified digestion time out of the starting amount of HA at time 0 expressed in percent of the starting HA amount) and the horizontal axis represents the degradation time in hours. In FIG. 12 the curve 70 represents the digestion results for the matrix obtained in Experiment May 2, 2002 and the curve labeled 72 represents the digestion results for Perlane® (Lot No. 7576). From the graph of FIG. 12 it may be seen that the matrix produced in Experiment May 2, 2002 has a resistance to hyaluronidase degradation which is far superior to the resistance of the tested commercial sample of Perlane®.

For example, after 5 hours of digestion practically all the Perlane® was digested, while approximately 68% of the sample of the matrix produced in EXPERIMENT 05/02/02 remained undigested. The results of the Perlane® digestion test are also summarized in TABLE 4B.

TABLE 4A

| | | Mass of | Amount of | Amount of | Hyaluronidase |
|---|---|---|---|---|---|
| Digestion | Sample | HA in | digested | remaining | Degradation |
| time | volume | sample | HA | HA | Resistance |
| [Hours] | [μL] | [mg] | [mg] | [mg] | [%] |
| 0 | 97.5 | 2.5 | 0.0 | 2.5 | 100.0 |
| 1 | 94.5 | 2.4 | 0.0 | 2.4 | 100.0 |
| 2 | 102.3 | 2.6 | 0.2 | 2.4 | 93.7 |
| 3 | 103.4 | 2.6 | 0.4 | 2.2 | 85.1 |
| 4 | 96.5 | 2.5 | 0.7 | 1.8 | 71.2 |
| 5 | 99.0 | 2.5 | 0.8 | 1.7 | 68.3 |

TABLE 4B

| | Perlane | Mass of | Amount of | Amount of | Hyaluronidase |
|---|---|---|---|---|---|
| Digestion | Sample | Perlane | digested | remaining | Degradation |
| time | volume | in sample | Perlane | Perlane | Resistance |
| [Hours] | [μL] | [mg] | [mg] | [mg] | [%] |
| 0 | 117.5 | 2.4 | 0.5 | 1.9 | 79.4 |
| 1 | 111.5 | 2.2 | 1.8 | 0.5 | 20.5 |
| 2 | 91.7 | 1.8 | 1.7 | 0.1 | 5.0 |
| 3 | 108.1 | 2.2 | 2.2 | 0.0 | 0.0 |
| 4 | 98.5 | 2.0 | 1.9 | 0.0 | 1.8 |
| 5 | 108.1 | 2.2 | 2.3 | 0.0 | 0.0 |

TABLE 5

| | Complex viscosity | |
|---|---|---|
| | \|η*\| in Pascal | |
| | (at an oscillating | |
| Experiment | frequency | Appearance of the resulting |
| Number | of 0.01 Hz) | material |
| 03/105/1 | 4673 | Hard gel |
| 03/105/2 | 8288 | Yellowish hard gel |
| 03/105/3 | — | Yellowish, hard pellets |
| 03/105/4 | — | Hard material |
| 03/105/5 | — | Hard material |
| 03/105/6 | — | Hard material |
| 03/114/1 | 7069 | Opaque gel |
| 03/114/2 | 6436 | Opaque gel |
| 03/114/3 | | Opaque gel |
| 03/114/4 | 6761 | Opaque gel |
| 03/140/1 | 72 | Transparent gel (1.5 mL) |
| 03/140/2 | 3691 | Yellowish gel (1.5 mL) |
| 03/140/3 | 2384 | Yellowish gel (1.5 mL) |
| 03/140/4 | 9 | Soft gel (3.7 mL) |
| 03/140/6 | 3135 | Soft, opaque gel (1.2 mL) |
| 03/110/1 | — | Soft gel (5.5 mL) |
| 03/110/2 | — | Hard yellow plates |
| 03/110/3 | — | Very firm gel (0.5 mL) |
| 03/110/4 | — | Hard gel |
| 03/131/2 | — | Firm yellow gel (0.5 mL) |
| 03/131/3 | 34 | Gel (3.2 mL) |
| 03/131/4 | — | Inhomogeneous yellow gel (0.8 mL) |
| 03/131/5 | 543 | Gel (4.5 mL) |
| 03/146/2 | 100 | Soft gel (0.9 mL) |
| 05/08/2 | 11 | Soft gel with opaque particles (5 mL) |
| 05/08/3 | 6 | Soft gel with opaque particles (1 mL) |
| 05/08/4 | 24.8 | Soft gel (10 mL) |
| 03/94/2 | 7740 | Hard gel (0.8 mL) |
| 04/55/1 | 1524 | Homogeneous white firm gel (5 mL) |
| 03/145/2 | 20230 | Red/brownish firm gel (3.7 mL) |
| 03/146/1 | 17870 | Opaque firm gel (2.7 mL) |
| 05/02/1 | 7810 | Light yellow opaque gel (4.5 mL) |
| 05/02/2 | 11540 | Light yellow transparent gel (4.5 mL) |
| 05/15/1 | 1326 | Slightly opaque gel (4.5 mL) |
| 05/18/1 | 45 | Soft gel (7.5 mL) |
| 05/22/1 | 21 | Soft gel (6 mL) |
| 05/22/2 | 83 | Soft gel (5 mL) |
| 05/22/3 | 55 | Soft gel (6 mL) |
| 05/22/4 | 6 | Soft gel (5 mL) |
| 05/23/1 | 10 | Soft gel |
| 05/23/1 | 0.2 | Liquid gel |
| 05/22/1 | 21 | Soft gel (6 mL) |
| 05/22/2 | 83 | Soft gel (5 mL) |
| 05/22/3 | 55 | Soft gel (6 mL) |
| 05/22/4 | 6 | Soft gel (5 mL) |
| 05/23/1 | 10 | Soft gel |
| 05/23/2 | 0.2 | Liquid gel |
| 09/95/1 | 2220 | Off-white gel |
| 09/95/2 | 2098 | Off-white gel |
| 09/95/3 | 2562 | Off-white gel |
| 09/95/4 | 4496 | Off-white gel |
| 09/102/1 | 573 | Off-white gel |
| 09/102/2 | 253 | Opaque gel |
| 09/102/3 | 47 | Opaque gel |
| 09/102/4 | 6934 | Off-white gel |
| 09/102/5 | 2321 | Opaque gel |
| 09/102/6 | 1038 | Opaque gel |

### Experiment 05/82/1

150 mg of AFHA II 150 were dissolved in 440 mL DI water and the solution transferred to a round bottom flask. 10 mg D(−)-fructose was dissolved in 10 mL saline. The resulting solution was mixed with the AFHA II 150 solution and the resulting mixture was slowly evaporated by rotation of the flask under vacuum. The concentrated mixture (approximately 2 mL) was incubated for 2 days at 37° C. under light vacuum. At the end of the incubation period, 30 mL saline were added to the contents of the flask and rotated for 1 hour without vacuum. The resulting gel was removed, filtered through a Whatman paper filter (No. 113) and brought to a final volume of 6 mL by diluting the gel with saline. The material was then sequentially extruded through 16G, 18G, 20G, 21G and 22G needles. Each extrusion step was repeated three times. The resultant particles were yellowish and had a firm consistency.

### Experiment Series 09/95/1-4

### Experiment 09/95/1

An aqueous sample of a solution of AFHA II 150 (1 mg/mL), containing a total amount of 200 mg AFHA II 150 was prepared. 1.2 mL of a solution of fibrillated porcine collagen (16.5 mg/mL) were added to the sample. 100 mg of D(−)-fructose dissolved in 10 mL saline were then added to the collagen/AFHA II 150 mixture. The resulting mixture was stirred for 1 min at 800 rpm with a turbine stirrer (A model R 1312 Turbine Stirrer commercially available from IKA®-Werke, GmbH & Co., Germany), transferred into a stainless steel tray and lyophilized. After lyophilization, the sample was covered with an ethanol/DI water mixture (90:10 v/v) and incubated at 37° C. for 6 hours. After the incubation, the material was washed three times with an ethanol/DI water mixture (90:10 v/v), the solvent was removed by draining the sample, and the sample was dried by lyophilization. 2 mL of saline were add to the lyophilized material and the mixture was incubated for 3 days at 37° C. At the end of the incubation period, the sample was extruded though a 16G needle, 4 mL of saline were added and the material was extruded again through a 18G and 20G needle.

### Experiment 09/95/2

The experiment was performed as described for experiment 09/95/1 above except that the amount of D(−)-fructose used was 130 mg.

### Experiment 09/95/3

The experiment was performed as described for experiment 09/95/1 above except that the amount of D(−)-fructose used was 160 mg.

### Experiment 09/95/4

The experiment was performed as described for experiment 09/95/1 above except that the amount of D(−)-fructose used was 100 mg and no collagen was added (this experiment was a control for AFHA II 150 cross-linked without collagen).

### Experiment Series 09/102/1-6

An aqueous solution of AFHA II 150 (at a concentration of 2.85 mg/mL of DI water) was used to prepare a sample, containing a total amount of 300 mg AFHA II 150. 1.8 mL of a solution of fibrillated collagen (having a concentration of 16.5 mg collagen/mL of fibrillation buffer) were added to samples 2-5 (of Experiments 09/102/2-5, respectively). 1.8 mL of fibrillation buffer were added to Sample 1 instead of the collagen solution (control with no collagen—Experiment 09/102/1). 5.0 mL of a solution of D(−)-fructose in saline (having a concentration of 40 mg D(−)-fructose per ml of saline) were added to each of the six samples and the samples were mixed. All the resulting reaction mixtures were stirred for 1 minute at 800 rpm with a turbine stirrer; transferred into separate stainless steel trays and lyophilized. After lyophilization, samples 1, 2 and 3 (of experiments 09/102/1, 09/102/2 and 09/102/3, respectively) were covered with an ethanol/DI water mixture (90:10 v:v) and incubated at 37° C. for 6 hours. Each of the samples1, 2 and 3 (of experiments 09/102/1, 09/102/2 and 09/102/3, respectively) was washed three times with an ethanol/DI water mixture (90:10 v:v), the solvent was removed by draining the samples and the samples were dried by lyophilization. Samples 4, 5 and 6 (of experiments 09/102/4, 09/102/5 and 09/102/6, respectively) were not washed.

2 mL of saline were added to each of the samples 1-6 and all of the samples were incubated for 3 days at 37° C. After the incubation, all the samples were extruded once though a 16 G needle. 4 mL saline were added to each of the extruded samples and each of the samples was sequentially extruded once through an 18G needle and once through a 20G needle.

The detailed quantities of materials and the reaction conditions used in each of the experiments of the EXPERIMENT SERIES 09/102/1-6 are given in TABLE 6 below.

TABLE 6

| | Amount | volume | | Incubation at | | Incubation |
|---|---|---|---|---|---|---|
| | of | of added | | 37° C. in | 3 washing | at 37° C. |
| | AFHA | fibrillated | Amount of | ethanol/saline | cycles in | in 2 mL of |
| Experiment | II 150 | collagen | D(—) fructose | mixture | ethanol/saline | saline |
| number | [mg] | [mL] | in saline (40 mg/mL) | (90:10) | mixture (90:10) | (days) |
| 09/102/1 | 150 | 0.0 mL | 200 mg | 6 hours | yes | 3 |
| 09/102/2 | 150 | 1.8 mL | 200 mg | 6 hours | yes | 3 |
| 09/102/3 | 150 | 3.6 mL | 200 mg | 6 hours | yes | 3 |
| 09/102/4 | 150 | 0.0 mL | 200 mg | — | no | 3 |
| 09/102/5 | 150 | 1.8 mL | 200 mg | — | no | 3 |
| 09/102/6 | 150 | 3.6 mL | 200 mg | — | no | 3 |

Some properties of the gels resulting from Experiments 09/102/1-6 are given in TABLE 5 above.

### Experiment Series 11/40/1, 2

The chitosan base used in experiments 11/40/1 and 11/40/2 described below is commercially available as Protasan UP B 80/200 from NovaMatrix FMC Biopolymer, Oslo, Norway.

### Experiment 11/40/1

An aqueous solution of AFHA II 150 (1.0 mg/mL) containing 300 mg AFHA II 150 was prepared. A solution containing 30 mg chitosan dissolved in 0.1 M HCl (pH 5—adjusted by adding fibrillation buffer) and 330 mg D(−)-fructose dissolved in 10 mL saline were added to the AFHA II 150 solution with mixing. The mixture was stirred for 1 minute at 800 rpm with a turbine stirrer, transferred into a stainless steel tray and lyophilized. After lyophilization, the sample was covered with an ethanol/DI water mixture (90:10 v:v) and incubated at 37° C. for 6 hours. The resulting material was washed three times with an ethanol/DI water mixture (90:10 v:v), the solvent was removed by draining and the sample was dried by lyophilization. 4 mL of saline were added to the lyophilized material and the material was incubated for 3 days at 37° C. At the end of the incubation, the sample was extruded though a 16 G needle, 8 mL saline were added and the mixture was again sequentially extruded through a 18G needle and a 20 G needle.

### Experiment 11/40/2

The experiment was performed as described for experiment 11/40/1 hereinabove except that the AFHA II 150 solution was mixed with 60 mg chitosan dissolved in 0.1 M HCl (pH 5—adjusted by adding fibrillation buffer) and 360 mg D(−) fructose dissolved in 10 mL saline. The resulting material was a gel having a firm consistency and an off-white/yellow color.

### Experiment Series 11/40/3-5

### Experiment 11/40/3

An aqueous solution of AFHA II 150 (1.0 mg/mL) containing 300 mg AFHA II 150 was prepared. A solution of 1.1 millimole (237 mg) of D(+)-glucosamine hydrochloride were dissolved in 10 mL saline was mixed with the aqueous AFHA II 150 solution. The mixture was stirred for 1 minute at 800 rpm with a turbine stirrer, transferred into a stainless steel tray and lyophilized. After lyophilization, the sample was covered with an ethanol/DI water mixture (90:10 v:v) and incubated at 37° C. for 6 hours. The resulting material was washed three times with an ethanol/DI water mixture (90:10 v:v), the solvent was removed by draining and the sample was dried by lyophilization. 4 mL of saline were added to the lyophilized material and the material was incubated for 3 days at 37° C. At the end of the incubation, the sample was extruded though a 16 G needle, 8 mL saline were added and the mixture was again sequentially extruded through a 18G needle and a 20 G needle. The resulting material was a gel having a firm consistency and an off-white/yellow color.

### Experiment 11/40/4

The experiment was performed as described for experiment 11/40/3 hereinabove, except that the reducing sugar used was 396 mg (1.1 millimole) of maltose monohydrate (instead of glucosamine hydrochloride). The resulting material was a gel having a firm consistency and an off-white/yellow color.

### Experiment 11/40/5

The experiment was performed as described for experiment 11/40/3 hereinabove, except that the reducing sugar used was 396 mg (1.1 millimole) of D(+)-lactose monohydrate (instead of D(+)-glucosamine hydrochloride). The resulting material was a gel having a firm consistency and an off-white/yellow color.

It is noted that while a limited number of reducing sugar types was used in the exemplary experiments disclosed hereinabove, many other types of reducing sugars and/or derivatives of reducing sugars may be used as cross-linkers for producing the cross-linked matrices of the present invention. Such reducing sugars may include, but are not limited to, an aldose, a ketose, a diose, a triose, a tetrose, a pentose, a hexose, a septose, an octose, a nanose, a decose, glycerose, threose, erythrose, lyxose, xylose, arabinose, ribose, allose, altrose, glucose, fructose, mannose, gulose, idose, galactose, talose, a reducing monosaccharide, a reducing disaccharide, a reducing trisaccharide, a reducing oligosaccharide, maltose, lactose, cellobiose, gentiobiose, melibiose, turanose, trehalose, isomaltose, laminaribiose, mannobiose and xylobiose. glyceraldehyde, ribose, erythrose, arabinose, sorbose, fructose, glucose, and combinations thereof.

Other types of reducing sugars which may be used for making the cross-linked matrices of the present invention are the reducing sugars and reducing sugar derivatives disclosed, inter alia, in U.S. Pat. Nos. 5,955,438, 6,346,515, and 6,682,760 and in Published International Patent Application WO 2003/049669 all which are all incorporated herein by reference in their entirety for all purposes.

It is further noted that in accordance with an embodiment of the invention, suitable reducing sugar derivatives may also be used for the cross-linking of the matrices of the present invention, such derivatives may include but are not limited to, D-ribose-5-phosphate, glucosamine, and any other type of other reducing sugar derivatives known in the art. Esters and salts of any of the above reducing sugar and their derivatives may also be used singly or in any suitable combination with the above disclosed reducing sugar types.

It is still further noted that in accordance with additional embodiments of the present invention the reducing sugar(s) used may be dextrorotatory, laevorotatory and mixtures of dextrorotatory and laevorotatory forms. Racaemic mixtures of one or more reducing sugars may also be used. Additionally, any reducing sugars which contain one or more asymmetric (chiral) carbon atoms may also be used in the methods and matrices of the present invention including various optically active isomeric forms (enantiomers) and/or any mixtures and combinations thereof.

It will be appreciated by those skilled in the art that in accordance with an additional embodiment of the present invention more than one reducing sugar may be used for cross-linking the amino-polysaccharides and/or amino-functionalized polysaccharides and/or any mixture of different amino-polysaccharides and/or amino-functionalized polysaccharides and/or any mixture of amino-polysaccharides and/or amino-functionalized polysaccharides with one or more proteins (and or any desired additives). For example, in accordance with a non-limiting example, AHFA I 150 may be cross-linked with as mixture of D(−)-ribose and D(+)-sorbose. Similarly, in accordance with another embodiment of the invention a mixture of chitosan and AHFA I 150 may be cross-linked in a mixture containing maltose, glucose and fructose. In yet another exemplary embodiment, a mixture of AHFA, collagen and heparin may be cross-linked with a mixture of cross-linkers including ribose, glucosamine and D-ribose-5-phosphate. These embodiments are given by way of example only and many other variations and modifications are possible by changing the number and type of polysaccharides used and the number and type of reducing sugars included in the cross-linking reaction mixture.

It will be appreciated by those skilled in the art that while the specific cross-linking reactions disclosed hereinabove make use of a limited exemplary range of solvents and solvent mixtures, many modifications and variations may be made in the solvent systems used in the cross-linking reactions of the present invention. Thus, the cross-linking reactions used to form the matrices of the present invention may be performed in aqueous solutions, buffered aqueous solutions, solutions including water and/or an aqueous buffered solution and one or more organic solvents, non-aqueous solutions including one or more non-aqueous solvents, and the like. As may be seen from the actual experiments disclosed hereinabove, the non-aqueous solvents used may be polar and/or hydrophilic and/or water miscible solvents but may also include various different non-polar, non-hydrophobic and solvent(s) which are not substantially miscible in water. In principle, any type of solvent system including any solvent or solvent combinations may be used for performing the cross-linking reactions of the present reactions provided that reasonable care is used in the selection of solvents.

For example, the solvents should preferably (but not obligatorily) not contain excessively chemically reactive groups or moieties that may adversely affect or interfere with the cross-linking reactions (unless any interfering side reactions are not undesirable, or are actually tolerable or even desired). Similarly, care should be taken in the choice of the type of solvent(s) used to avoid undesired denaturing of any proteins and/or polypeptides which are being cross-linked together with the amino-polysaccharides and/or amino-functionalized polysaccharides. Bearing in mind such precautions, almost any type of solvent or solvent mixture or solvent system may be used for performing the cross-linking reactions of the present invention.

Thus, the matrices of the present invention may be formed by cross-linking any suitable combination of amino-polysaccharides and/or amino-functionalized polysaccharides and/or any mixture of different amino-polysaccharides and/or amino-functionalized polysaccharides and/or any mixture of amino-polysaccharides and/or amino-functionalized polysaccharides with one or more proteins with any desired combination of reducing sugars and/or reducing sugar derivatives. All such combinations and permutations are considered to be within the scope of the present invention. The use of such various combinations may be advantageously used for fine tuning of the chemical and/or physical and/or rheological and/or biological properties of the resulting cross-linked matrices, to adapt the matrices for any desired application. The properties of the resulting matrices may thus depend, inter alia, on the number and properties of the amino-polysaccharides and/or amino functionalized polysaccharides used, the number and type of proteins used (if used), the number and type of the cross-linking reducing sugars, and the properties of any other additive included in the matrix. It is also noted that the matrix properties may also be affected, inter alia, by the reaction conditions, the reaction temperature, the pH, the type of solvent or solvents used and the presence or absence of any additives present in the reaction mixture and/or added to the matrices after the cross-linking.

It is noted that the solvent(s) used in the cross-linking reaction mixture may include at least one ionizable salt (such as, but not limited to, the NaCl used in the Saline solutions of experiment 09/95/1 and in experiments 09/102/1-6, or the PBS used in experiments 2, 12/1 and 37/1-3 and in other experiments as disclosed in detail hereinabove). The ionizable salt(s) may be useful for controlling the ionic strength of said solution, and may be advantageous in implementing methods for forming composite matrices including proteins in cases where the proteins are sensitive to the ionic strength of the reaction solution. It is noted that any suitable ionizable salt(s) known in the art may be used to control the ionic strength of the reaction solution as is well known in the art. Some non-limiting examples of ionizable salts which may be used include various alkali metal salts, alkali metal halides, various different metallic sulfates and/or phosphates, various different ammonium salts and the like, as is known in the art. However, any other suitable type of ionizable salt(s) known in the art may also be used in the cross-linking reactions of the present invention.

It is noted that the products of the novel cross-linking reactions described hereinabove may be used to obtain a variety of different cross-linked polysaccharide based matrices and polysaccharide/protein based composite matrices. Such matrices may be obtained as, or may be suitably processed (by suitable use of molds and/or compression, and/or drying, and/or lyophylization and/or any other method known in the art for forming solid or semi-solid articles from such matrices) to provide solid forms of matrices in any desired shape, and/or any form of injectable preparation, including but not limited to injectable and non-injectable suspensions of matrix particles, microspheres, microparticles of any desire size and shape. Solid forms of the matrices may include, but are not limited to, sheets, tubes, membranes, sponges, flakes, gels, beads, microspheres, microparticles and other related geometrical forms made of any of the polysaccharide based matrix types disclosed hereinabove (including but not limited to polysaccharide/protein composite matrices) that may be obtained by cross-linking using the glycation methods of the present invention.

It is noted that the products of the novel cross-linking reactions described hereinabove (including both the sugar cross-linked polysaccharides and the sugar cross-linked composite protein/polysaccharide matrices) may be further processed and/or treated and/or modified by subjecting the cross-linked matrices to further treatment and/or one or more processing steps. Such treatments and/or modifications may include but are not limited to, drying, freeze-drying, dehydration, critical point drying, molding in a mold (to form shaped articles), sterilization, homogenization (to modify or improve flow properties and injectability of the matrices), mechanical shearing (to modify rheological properties and ease of injecting), irradiation by ionizing radiation (for sterilization purposes and/or for performing additional cross-links and/or for other purposes), irradiation by electromagnetic radiation (for sterilization purposes and/or for performing additional cross-links and/or for other purposes), mixing with a pharmaceutically acceptable vehicle (such as, for example, for forming an injectable preparation for tissue bulking, and/or tissue augmentation an/or other purposes), sterilization by thermal means (autoclaving and the like), sterilization by chemical means (such as, but not limited to sterilization using hydrogen peroxide, ozone, ethylene oxide, and the like), impregnation with an additive and/or any combinations of such processing steps.

Furthermore, any suitable combinations of the above disclosed additional treatments or processing steps may be used, in any suitable sequence, to provide any desired modified, and/or dried, and or shaped articles and/or preparations of the novel sugar cross-linked matrices disclosed herein. All the above described treatment methods are well known in the art and are therefore not described in detail hereinafter.

It is further noted that the composite matrices of the present invention are not limited to the use of any particular type of collagen. Rather, any desired type of collagen, including but not limited to, native collagen, fibrillar collagen, fibrillar atelopeptide collagen, telopeptide containing collagen, lyophylized collagen, collagen obtained from animal sources, human collagen, mammalian collagen, recombinant collagen, pepsinized collagen, reconstituted collagen, bovine atelopeptide collagen, porcine atelopeptide collagen, collagen obtained from a vertebrate species, recombinant collagen, genetically engineered or modified collagen, collagen types I, II III, V, XI, XXIV, fibril-associated collagens types IX, XII, XIV, XVI, XIX, XX, XXI, XXII and XXVI, collagens types VIII and X, type IV collagens, type VI collagen, type VII collagen, type XIII, XVII, XXIII and XXV collagens, type XV and XVIII collagens, artificially produced collagen manufactured by genetically modified eukaryotic or prokaryotic cells or by genetically modified organisms, purified collagen and reconstituted purified collagen, particles of fibrillar collagen, fibrillar reconstituted atelopeptide collagen, artificially produced collagen manufactured by genetically modified eukaryotic or prokaryotic cells or by genetically modified organisms, purified collagen and reconstituted purified collagen, particles of fibrillar collagen, fibrillar reconstituted atelopeptide collagen, collagen purified from cell culture medium, collagen derived from genetically engineered plants, fragments of collagen, proto-collagen and any combinations of the above listed collagen types may be used in forming the composite matrices of the present invention, as disclosed hereinabove.

It will be appreciated by those skilled in the art that the composite matrices disclosed in the present application are not limited to the use of collagen and cytochrome C as experimentally demonstrated hereinabove. Rather, the composite matrices of the present invention may include matrices including in addition to the amino-polysaccharides and/or amino-functionalized polysaccharides any suitable type of protein(s) and/or polypeptides (natural or synthetic) which is cross-linkable to the amino-polysaccharides and/or amino-functionalized polysaccharides by one or more reducing sugar cross-linker and/or a reducing sugar derivative cross-linker. Such cross-linkable protein or polypeptide may include, but are not limited to, collagen, a protein selected from the collagen superfamily, extra-cellular matrix proteins, enzymes, structural proteins, blood derived proteins, glycoproteins, lipoproteins, natural proteins, synthetic proteins, hormones, growth factors, cartilage growth promoting proteins, bone growth promoting proteins, intracellular proteins, extracellular proteins, membrane proteins, elastin, fibrin, fibrinogen, and various different combinations thereof.

In accordance with one aspect of the present invention, the cross-linked polysaccharide matrices of the present invention may be formulated into suitable injectable formulations, with or without suitable pharmaceutical additives and/or pharmaceutically acceptable vehicle(s). Such injectable preparations may be packaged in a suitable syringe (with or without a suitable needle). Such pre-filled, pre-sterilized syringes may be useful in a variety of cosmetic and medical applications, such as, but not limited to, wrinkle smoothing applications, tissue augmentation, tissue bulking, and the like.

According to an additional embodiment of the invention, the matrices of the present invention may be chemically and/or physically and/or biologically modified with agents and substances such as, but not limited to, pharmaceuticals, drugs, proteins, polypeptides, anesthetic agents, anti-bacterial agents, anti-microbial agents, anti-viral agents, anti-fungal agents, anti-mycotic agents, anti-inflamatory agents, glycoproteins, proteoglycans, glycosaminoglicans, various extracellular matrix components, hormones, growth factors, transforming factors, receptors or receptor complexes, natural polymers, synthetic polymers, DNA, RNA, olygonucleotides, a drug, a therapeutic agent, an anti-inflammatory agent, glycosaminoglicans, proteoglycans, morphogenic proteins glycoproteins, mucoproteins, mucopolysaccharides, matrix proteins, growth factors, transcription factors, anti-inflammatory agents, proteins, peptides, hormones, genetic material for gene therapy, a nucleic acid, a chemically modified nucleic acid, an oligonucleotide, ribonucleic acid, deoxyribonucleic acid, a chimeric DNA/RNA construct, DNA or RNA probes, anti-sense DNA, anti-sense RNA, a gene, a part of a gene, a composition including naturally or artificially produced oligonucleotides, a plasmid DNA, a cosmid DNA, viral and non-viral vectors required for promoting cellular uptake and transcription, a glycosaminoglycan, chondroitin 4-sulfate, chondroitin 6-sulfate, keratan sulfate, dermatan sulfate, heparin, heparan sulfate, hyaluronan, a lecitin rich interstitial proteoglycan, decorin, biglycan, fibromodulin, lumican, aggrecan, syndecans, beta-glycan, versican, centroglycan, serglycin, a fibronectin, fibroglycan, chondroadherins, fibulins, thrombospondin-5, an enzyme, an enzyme inhibitor, an antibody, and by any combinations of the above materials and/or any other type of property modifying agent or substance known in the art. Such agents or substances may be added to the matrices after the cross-linking has been completed. Additionally or alternatively, agent(s) or substance(s) may be added to the reaction mixture prior to cross-linking and the cross-linking reaction may then be performed in the presence of the agent (s) or substance(s) to incorporate and/or to cross-link the agent(s) or the substance(s) within the formed cross-linked matrix to change the matrix properties.

Such added substances may be covalently linked to the polysaccharide matrix by any suitable cross-linking agents, as is well known in the art. Alternatively or additionally, such modifying substances may be included in the reaction mixture during the cross-linking processes described herein and may thus be trapped within or incorporated into the cross-linked polysaccharide based matrices or composite matrices.

It will be appreciated by those skilled in the art that the cross-linked matrices described herein may be further modified by subjecting the matrices to any chemical or biological modifiers known in the art. For example, some or all of the free functional groups such as, but not limited to amino groups and/or carboxy groups, and/or hydroxyl groups remaining on the cross-linked matrix components after the cross-linking may be chemically or enzymatically treated to chemically introduce other chemical groups or moieties (such as, but not limited to amino groups and/or carboxy groups, and/or hydroxyl groups and/or nitro-groups, and/or chloro- and/or bromo- and/or iodo-groups, and/or peroxo-groups and/or periodo-groups and/or perchloro-groups, and/or any other chemical groups and/or chemical moieties the like) to further modify such groups in order to further control the matrix properties. Examples of such possible post-cross-linking modifications may include but are not limited to, esterification of free hydroxyl or carboxy groups present on the polysaccharide backbone of the cross-linked matrix or on the protein backbone of any included cross-linked protein or polypeptide of a composite matrix, acetylation of any free amino groups on polysaccharide or polypeptide backbones, or any other type of functional group chemical or enzymatic modification reactions known in the art. The chemistry of such modifications is well known in the art and is therefore not disclosed in detail hereinafter.

Such functional group modifications may be useful for further modifying and fine tuning the matrix properties (such as, but not limited to, hydrophobicity, hydrophillicity, net charge at various selected pH levels, matrix porosity, matrix water absorbing capacity, resistance to enzymatic degradation in-vivo and/or in vitro and the like) to adapt the matrix for specific desired applications. It should be born in mind that if the matrices which are being modified are intended for uses which require biocompatibility, care should be exercised in the selection of the chemical groups being modified and in the nature of any chemical groups which are being introduced into the matrices' structure to ensure a sufficient degree of biocompatibility. However, in other applications of the matrices which do not require a high degree of biocompatibility, many of the groups listed above and any other chemical groups known in the art (such as, but not limited to azo-groups, azido-groups nitroso-groups, and the like) may be introduced into the structure of the matrices to provide further modification of the matrix structure and properties.

In accordance with an additional embodiment of the matrices of the present invention, living cells may be added to any of the cross-linked matrices described hereinabove or prepared using the methods disclosed herein. The living cells may be added during or after the cross-linking, to form a cross-linked matrix containing one or more living cell included or embedded in the matrix.

In accordance with an additional embodiment of the matrices of the present invention, the living cells included in the matrices may be vertebrate chondrocytes, osteoblasts, osteoklasts, vertebrate stem cells, embryonal stem cells, adult tissue derived stem cells, vertebrate progenitor cells, vertebrate fibroblasts, cells genetically engineered to secrete one or more of matrix proteins, glycosaminoglicans, proteoglycans, morphogenic proteins, growth factors, transcription factors, anti-inflammatory agents, proteins, hormones, peptides, one or more types of living cells engineered to express receptors to one or more molecules selected from the group consisting of proteins, peptides, hormones, glycosaminoglicans, proteoglycans, morphogenic proteins, growth factors, transcription factors, anti-inflammatory agents, glycoproteins, mucoproteins, and mucopolysaccharides. Combinations of several types of different cells may also be included in the matrices of the present invention.

In accordance with different embodiments of the present invention, the cross-linked polysaccharide based matrices obtained by the methods of the present application may be suitable for use in different applications such as, but not limited to, matrix scaffolds useable in tissue engineering (for in-vivo and in-vitro applications), controlled delivery systems for pharmaceuticals and biologics (biologically active proteins, genes, gene vectors and the like), membranes for guided tissue and bone regeneration, injectable and/or implantable bulking agents and/or prosthetic devices for tissue augmentation and/or for cosmetic use (such as, but not limited to, injectable preparations for wrinkle filling and other cosmetic and aesthetic purposes), envelopes for anchoring natural and/or reconstructed and/or artificial organs, filler material for the preparation of artificial tissues or organs such as but not limited to artificial breast, and as a component of composite materials comprising the cross-linked polysaccharides of the present invention combined with other natural or artificial polymeric structures, materials and/or matrices or with other natural or synthetic organic and inorganic compounds and/or polymers and/or combinations of all of the above substances.

It will be appreciated by those skilled in the art that while the buffer used in many of the reactions and sample preparations disclosed above was phosphate buffered saline (PBS), this is by no means obligatory to practicing the invention. Thus, many different types of buffers and or buffered solutions and buffered solvents may be used to perform the material preparation procedures and/or the cross-linking reactions for preparing the polysaccharide based matrices and/or the polysaccharide/protein based composite matrices of the present invention. For example, other exemplary buffers that may be used in the preparations and cross-linking reactions of the present invention may include but are not limited to citric acid/citrate buffers, 2-(N-Morpholino)ethanesulfonic acid (MES), 2-Bis(2-hydroxyethyl)amino-2-(hydroxymethyl)-1,3-propanediol (BIS-TRIS), piperazine-N,N′-bis(2-ethanesulfonic acid) (PIPES), 3-(N-Morpholino)propanesulfonic acid (MOPS), 4-(2-Hydroxyethyl)piperazine-1-ethanesulfonic acid (HEPES), and many other types of buffers known in the art. However, care should be taken in the choice of buffer compositions (if used) to ensure that the buffers do not include active chemical groups or moieties that may interfere with the cross-linking reactions described hereinabove. Such buffers and the considerations of their selection for use are well known in the art, are extensively described in the literature and are therefore not disclosed in detail herein.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

It is noted that some of the above described embodiments may describe the best mode contemplated by the inventors and therefore may include structure, acts or details of structures and acts that may not be essential to the invention and which are described as examples. Structure compounds and acts described herein are replaceable by equivalents which perform the same function, even if the structure or acts are different, as known in the art. Therefore, the scope of the invention is limited only by the elements and limitations as used in the claims. When used in the following claims, the terms “comprise”, “include”, “have” and their conjugates mean “including but not limited to”.

What is claimed is:

1. A method for preparing cross-linked polysaccharides, the method comprising reacting at least one polysaccharide selected from an amino-polysaccharide, an amino-functionalized polysaccharide and combinations thereof with at least one reducing sugar, to form a cross-linked polysaccharide.

2. The method according to claim 1 wherein said at least one polysaccharide is selected from a naturally occurring amino-polysaccharide, a synthetic amino-polysaccharide, an amino heteropolysaccharide, an amino homopolysaccharide, amino-functionalized polysaccharides and derivatized forms and esters and salts thereof, amino-functionalized hyaluronic acid and derivatized forms and esters and salts thereof, an amino-functionalized hyaluronan and derivatized forms and esters and salts thereof, chitosan and derivatized forms thereof and esters and salts thereof, heparin and derivatized forms and esters and salts thereof, amino functionalized glycosaminoglycans and derivatized forms and esters and salts thereof, and any combinations thereof.

3. The method according to claim 1 wherein said at least one reducing sugar is selected from an aldose, a ketose, a derivative of an aldose, a derivative of a ketose and any combinations thereof.

4. The method according to claim 1 wherein said at least one reducing sugar is selected from a diose, a triose, a tetrose, a pentose, a hexose, a septose, an octose, a nanose, a decose, and combinations thereof.

5. The method according to claim 1 wherein said at least one reducing sugar is selected from glycerose, threose, erythrose, lyxose, xylose, arabinose, ribose, allose, altrose, glucose, fructose, mannose, gulose, idose, galactose and talose.

6. The method according to claim 1 wherein said at least one reducing sugar is selected from a reducing monosaccharide, a reducing disaccharide, a reducing trisaccharide, a reducing oligosaccharide, derivatized forms of oligosaccharides, derivatized forms of monosaccharides, esters of monosaccharides, esters of oligosaccharides, salts of monosaccharides, salts of oligosaccharides and any combinations thereof.

7. The method according to claim 6 wherein said reducing disaccharide is selected from the group consisting of maltose, lactose, cellobiose, gentiobiose, melibiose, turanose, trehalose, isomaltose, laminaribiose, mannobiose and xylobiose.

8. The method according to claim 1 wherein said at least one reducing sugar is selected from glyceraldehyde, ribose, erythrose, arabinose, sorbose, fructose, glucose, D-ribose-5-phosphate, glucosamine, and combinations thereof.

9. The method according to claim 1 wherein said at least one reducing sugar is selected from a Dextrorotatory form of said at least one reducing sugar, a Laevorotatory form of said at least one reducing sugar and a mixture of Dextrorotatory and Laevorotatory forms of said at least one reducing sugar.

10. The method according to claim 1 wherein said reacting comprises incubating said at least one polysaccharide in a solution comprising at least one solvent and said at least one reducing sugar, to form said cross-linked polysaccharide.

11. The method according to claim 10 wherein said solution is a buffered solution including at least one buffer.

12. The method according to claim 11 wherein said at least one solvent is an aqueous buffered solvent including at least one buffer for controlling the pH of said solution.

13. The method according to claim 11 wherein said at least one solvent is an aqueous solvent including at least one ionizable salt for controlling the ionic strength of said solution.

14. The method according to claim 10 wherein said at least one solvent comprises at least one solvent selected from the group consisting of an organic solvent, an inorganic solvent, a polar solvent, a non-polar solvent, a hydrophilic solvent, a hydrophobic solvent, a solvent miscible in water, a non-water miscible solvent and combinations thereof.

15. The method according to claim 10 wherein said at least one solvent comprises water and at least one additional solvent selected from a hydrophilic solvent, a polar solvent, a solvent miscible in water and combinations thereof.

16. The method according to claim 10 wherein said at least one solvent is selected from the group consisting of water, phosphate-buffered saline, ethanol, 2-propanol, 1-butanol, 1-hexanol, acetone, ethyl acetate, dichloromethane, diethyl ether, hexane, toluene, and combinations thereof.

17. The method according to claim 1 wherein said reacting also includes adding at least one protein or polypeptide having cross-linkable amino groups to said at least one polysaccharide and said at least one reducing sugar to form a composite cross-linked matrix.

18. The method according to claim 17 wherein said at least one protein or polypeptide having cross-linkable amino groups is selected from collagen, a protein selected from the collagen superfamily, extra-cellular matrix proteins, enzymes, structural proteins, blood derived proteins glycoproteins, lipoproteins, natural proteins, synthetic proteins, hormones, growth factors, cartilage growth promoting proteins, bone growth promoting proteins, intracellular proteins, extracellular proteins, membrane proteins, elastin, fibrin, fibrinogen and any combinations thereof.

19. The method according to claim 18 wherein said collagen is selected from, native collagen, fibrillar collagen, fibrillar atelopeptide collagen, telopeptide containing collagen, lyophylized collagen, collagen obtained from animal sources, human collagen, mammalian collagen, recombinant collagen, pepsinized collagen, reconstituted collagen, bovine atelopeptide collagen, porcine atelopeptide collagen, collagen obtained from a vertebrate species, recombinant collagen, genetically engineered or modified collagen, collagen types I, II III, V, XI, XXIV, fibril-associated collagens types IX, XII, XIV, XVI, XIX, XX, XXI, XXII and XXVI, collagens types VIII and X, type IV collagens, type VI collagen, type VII collagen, type XIII, XVII, XXIII and XXV collagens, type XV and XVIII collagens, artificially produced collagen manufactured by genetically modified eukaryotic or prokaryotic cells or by genetically modified organisms, purified collagen and reconstituted purified collagen, particles of fibrillar collagen, fibrillar reconstituted atelopeptide collagen, collagen purified from cell culture medium, collagen derived from genetically engineered plants, fragments of collagen, proto-collagen and any combinations thereof.

20. The method according to claim 1 wherein said reacting includes adding at least one additive to said at least one polysaccharide and said at least one reducing sugar to form a cross-linked matrix containing said at least one additive.

21. The method according to claim 20 wherein said at least one additive is selected from pharmaceuticals, drugs, proteins, polypeptides, anesthetic agents, anti-bacterial agents, anti-microbial agents, anti-viral agents, anti-fungal agents, anti-mycotic agents, anti-inflamatory agents, glycoproteins, proteoglycans, glycosaminoglicans, various extracellular matrix components, hormones, growth factors, transforming factors, receptors or receptor complexes, natural polymers, synthetic polymers, DNA, RNA, olygonucleoytyides, a drug, a therapeutic agent, an anti-inflammatory agent, glycosaminoglicans, proteoglycans, morphogenic proteins glycoproteins, mucoproteins, mucopolysaccharides, matrix proteins, growth factors, transcription factors, anti-inflammatory agents, proteins, peptides, hormones, genetic material for gene therapy, a nucleic acid, a chemically modified nucleic acid, an oligonucleotide, ribonucleic acid, deoxyribonucleic acid, a chimeric DNA/RNA construct, DNA or RNA probes, anti-sense DNA, anti-sense RNA, a gene, a part of a gene, a composition including naturally or artificially produced oligonucleotides, a plasmid DNA, a cosmid DNA, viral and non-viral vectors required for promoting cellular uptake and transcription, a glycosaminoglycan, chondroitin 4-sulfate, chondroitin 6-sulfate, keratan sulfate, dermatan sulfate, heparin, heparan sulfate, hyaluronan, a lecitin rich interstitial proteoglycan, decorin, biglycan, fibromodulin, lumican, aggrecan, syndecans, beta-glycan, versican, centroglycan, serglycin, a fibronectin, fibroglycan, chondroadherins, fibulins, thrombospondin-5, an enzyme, an enzyme inhibitor, an antibody, and any combinations thereof.

22. The method according to claim 1 further including adding one or more living cells to said at least one polysaccharide and said at least one reducing sugar before, during or after said cross-linking, to form a cross-linked matrix containing at least one live cell embedded in said matrix.

23. The method according to claim 22 wherein said one or more living cells are selected from vertebrate chondrocytes, osteoblasts, osteoklasts, vertebrate stem cells, embryonal stem cells, adult tissue derived stem cells, vertebrate progenitor cells, vertebrate fibroblasts, cells genetically engineered to secrete one or more of matrix proteins, glycosaminoglicans, proteoglycans, morphogenic proteins, growth factors, transcription factors, anti-inflammatory agents, proteins, hormones, peptides, one or more types of living cells engineered to express receptors to one or more molecules selected from the group consisting of proteins, peptides, hormones, glycosaminoglicans, proteoglycans, morphogenic proteins, growth factors, transcription factors, anti-inflammatory agents, glycoproteins, mucoproteins, and mucopolysaccharides, and any combinations thereof

24. The method according to claim 1 further including subjecting said cross-linked polysaccharide to a treatment selected from drying, freeze-drying, dehydration, critical point drying, molding, sterilization, homogenization, mechanical shearing, irradiation by ionizing radiation, irradiation by electromagnetic radiation, mixing with a pharmaceutically acceptable vehicle, impregnation with an additive and combinations thereof.

25. A cross-linked polysaccharide prepared by the method of claim 1.

26. A method for preparing cross-linked polysaccharides, the method comprising the steps of:
reacting a polysaccharide with one or more reactants to form a derivatized form of said polysaccharide, said derivatized form contains one or more amino groups; and
cross-linking said derivatized polysaccharide with at least one reducing sugar to form a cross-linked polysaccharide.

27. The method according to claim 26 wherein said amino groups are selected from primary amino groups and secondary amino groups.

28. The method according to claim 26 wherein said one or more reactants comprise a carbodiimide.

29. The method according to claim 26 wherein said one or more reactants comprise a carbodiimide in the presence of adipic acid dihydrazide.

30. The method according to claim 28 wherein said carbodiimide is 1-ethyl-3-(dimethyl aminopropyl)carbodiimide hydrochloride.

31. The method according to claim 26 wherein said at least one reducing sugar is selected from an aldose, a ketose, and combinations thereof.

32. The method according to claim 26 wherein said at least one reducing sugar is selected from glyceraldehyde, ribose, erythrose, arabinose, sorbose, fructose, glucose, D-ribose-5-phosphate, glucosamine, a diose, a triose, a tetrose, a pentose, a hexose, a septose, an octose, a nanose, a decose, glycerose, threose, erythrose, lyxose, xylose, arabinose, ribose, allose, altrose, glucose, fructose, mannose, gulose, idose, galactose, talose, a reducing monosaccharide, a reducing disaccharide, a reducing trisaccharide, a reducing oligosaccharide, derivatized forms of oligosaccharides, derivatized forms of monosaccharides, esters of monosaccharides, esters of oligosaccharides, salts of monosaccharides, salts of oligosaccharides, maltose, lactose, cellobiose, gentiobiose, melibiose, turanose, trehalose, isomaltose, laminaribiose, mannobiose and xylobiose, and combinations thereof.

33. A cross-linked polysaccharide prepared by the method of claim 24.

34. A method for preparing a composite cross-linked matrix, the method comprises cross-linking with at least one reducing sugar at least one polysacharide selected from an amino-polysaccharide, an amino-functionalized polysaccharide and combinations thereof in the presence of at least one cross-linkable protein to form said composite cross-linked matrix.

35. A cross-linked composite matrix prepared by the method of claim 34.
